(19) United States
(12) Patent Application Publication
(10) Pub. No.: US 2025/0188428 A1
(43) Pub. Date: Jun. 12, 2025

(54) NOVEL IN VITRO AND IN VIVO ENRICHMENT STRATEGY TARGETING LYMPHOCYTES DERIVED FROM VECTOR TRANSDUCED HSCS FOR THERAPY OF DISORDERS

(71) Applicants: The Medical College of Wisconsin, Inc., Milwaukee, WI (US); University Health Network, Toronto (CA); Washington University in St. Louis, St. Louis, MO (US)

(72) Inventors: Jeffrey A. Medin, Shorewood, WI (US); John F. Dipersio, St. Louis, MO (US); Murtaza S. Nagree, Milwaukee, WI (US)

(21) Appl. No.: 19/064,250

(22) Filed: Feb. 26, 2025

Related U.S. Application Data
(63) Continuation of application No. 18/179,876, filed on Mar. 7, 2023, now abandoned.
Application No. 18/179,876 is a continuation of application No. 16/619,883, filed on Dec. 5, 2019, now Pat. No. 11,597,917.
(60) Provisional application No. 62/516,022, filed on Jun. 6, 2017.
(60) Provisional application No. 62/664,671, filed on Apr. 30, 2018.

(51) Int. Cl. C12N 9/04; A61K 9/00; A61K 35/28; A61P 43/00; C12N 5/0789; C12N 7/00; C12N 9/24; C12N 9/40; C12N 9/80; C12N 15/86

(52) U.S. Cl. CPC C12N 9/0006; A61K 9/0019; A61K 35/28; A61P 43/00; C12N 5/0647; C12N 7/00; C12N 9/2465; C12N 9/2477; C12N 9/80; C12N 15/86; C12Y 101/01205; C12Y 302/0102; C12Y 302/01022; C12Y 302/01045; C12Y 305/01023; C12N 2510/02; C12N 2740/15043

(57) ABSTRACT

The present invention is related to a dual promoter lentiviral vector and methods of use for the treatment of diseases and disorders, specifically lysosomal storage disorders.

### CROSS-REFERENCE TO RELATED APPLICATIONS

This Application is a continuation of U.S. application Ser. No. 18/179,876, with filing date of Mar. 7, 2023, which is a continuation of U.S. application Ser. No. 16/619,833 with filing date of Dec. 5, 2019, now U.S. Pat. No. 11,597,917, which is the U.S. National Stage of PCT/US2018/036292 with international filing date of Jun. 6, 2018, which claims priority to U.S. Provisional Application No. 62/515,022 filed on Jun. 6, 2017, and U.S. Provisional Application No. 62/664,671 filed on Apr. 30, 2018. The contents of each of the above-referenced applications are incorporated by reference in their entireties.

### STATEMENT REGARDING FEDERALLY SPONSORED RESEARCH

N/A.

### SEQUENCE LISTING

A Sequence Listing accompanies this application and is submitted as an XML file of the sequence listing named “650053_00657.xml” which is 81,776 bytes in size and was created on Mar. 7, 2023. The sequence listing is electronically submitted via Patent Center with the application and is incorporated herein by reference in its entirety.

### BACKGROUND OF THE INVENTION

The field of the invention is related to treatment of diseases and disorders associated with a single altered protein or enzyme, in particular diseases that can be treated with engrafted hematopoietic cells, for example, lysosomal storage disorders such as Fabry disease and Gaucher disease which are associated with dysregulation or deficiency of a single protein or in some instances a single protein combined with a co-activator protein.

### Lysosomal Storage Disorders

Lysosomes are membrane-bound organelles in animal cells that contain more than 60 different enzymes capable of digesting nearly any biomolecule. They carry many critical biological functions, including acting as the cell's waste disposal system by digesting unwanted materials in the cytoplasm, both from outside of the cell and obsolete components inside the cell. Lysosomal Storage Disorders (LSDs, lysosomal storage diseases) are a group of more than 60 rare inherited metabolic disorders that result from lysosome dysfunction, usually as a consequence of a deficiency in a single enzyme required for the intracellular digestion of lipids, glycoproteins, or mucopolysaccharides. As a result of such deficiencies, the molecules that would normally be degraded accumulate inside the cell, leading to dysfunction or death of the cell.

### Fabry Disease

Fabry disease is a LSD resulting from a deficiency in the enzyme α-galactosidase A (, α-gal A encoded by the AGA transgene), an enzyme that hydrolyses α-galactose from glycosphingolipids, in particular globotriaosylceramide (Gb₃). Sphingolipids play an important role in signal transmission and cell recognition, and sphingolipidoses can impact all tissues. Currently, enzyme replacement therapy (ERT) is used to treat Fabry disease, and with ERT, patients with Fabry disease may live well into adulthood. However, ERT requires lengthy intravenous infusions of α-gal A administered every few weeks, usually at an outpatient center. In addition, Fabry patients often require treatments for pain, gastrointestinal dysfunction, arrhythmias and other heart problems, as well as blood thinners and blood pressure medications to protect kidney function. Although Fabry disease is relatively rare—there are about 4000 patients in the US—treatment costs are on the order of $300,000/year/patient, or $1.2B/y for all US patients.

### Genetic Modification of Hematopoietic Stem Cells From Fabry Patients

Prior work by the inventor related to the use of a cellular therapy for Fabry disease. The method is based on the use of hematopoietic stem cells (HSCs), which are “multipotent” cells present in bone marrow that can differentiate into all blood cell types. A method of genetically modifying HSCs taken from Fabry patients was developed by “transducing” the cells with a recombinant lentivirus vector carrying the transgene for the α-gal A enzyme. Upon re-introduction of the patient's modified cells back into the patient (an “autologous” graft), the genetically modified stem cells will populate all downstream lineages of the hematopoietic system and then circulate throughout the body. The modified cells secrete a form of α-gal A with a molecular “tag” (mannose 6-phosphate) which allows uncorrected “bystander” cells in the patient to take up and transport α-gal A intracellularly into their lysosomes, where it can compensate for the patient's α-gal A deficiency, and effectively degrade the accumulated sphingolipids. This method is undergoing clinical trials in Canada (ClinicalTrials.gov #NCT02800070).

However, systemic expression of α-gal A using this method is relatively low, in part because of a key step in the process. It is only possible to obtain a relatively small number of HSCs from patients as HSCs are relatively rare cells, and it is also difficult to get a high level of transduction in HSCs. These limitations may constrain ability of these cells to adequately increase the systemic levels of α-gal A within a subject. Increasing the number of cells producing α-gal A will in turn increase the systemic α-gal A levels. Thus, there is a need to develop methods of increasing the number of α-gal A-expressing cells after HSC engraftment to subsequently increase systemic levels of α-gal A for treatment of Fabry disease.

### Gaucher Disease

Gaucher disease is an autosomal recessive inherited disorder caused by mutations in the GBA transgene that leads to a reduction or elimination of activity of beta-glucocerebrosidase (GBA protein). This enzyme breaks down glucocerebroside into a sugar (glucose) and a simpler fat-like molecule (ceramide). Without enough of this enzyme, glucocerebroside and related substances can build up to toxic levels within cells, including the spleen, liver and bone tissue. Tissues and organs are damaged by the abnormal accumulation and storage of these substances, causing the characteristic features of Gaucher disease.

Traditional treatment for Gaucher disease is enzyme replacement therapy. However, better methods of treating such a disease are needed.

### SUMMARY OF THE INVENTION

The present invention overcomes the aforementioned drawbacks by providing a method of enriching for hematopoietic cells in vitro, ex vivo and in vivo that express the desired transgene, for example, but not limited to, AGA transgene for Fabry Disease or GBA transgene for Gaucher disease.

The inventors in the present invention have engineered a “dual promoter” lentivirus vector for expression of a protein from a transgene of interest, for example, but not limited to, α-gal A or GBA protein, and an enzyme, IMPDH2(IY), that confers mycophenolic acid (MPA) resistance to transduced B-and T-cells in the white cell population. MPA, which is delivered in vivo as the FDA approved drug mycophenolate mofetil (MMF), inhibits an enzyme needed for the growth of T cells and B cells. MMF is used routinely in the clinic as an immunosuppressive agent for a variety of indications (e.g., to prevent organ transplant rejection) with a low incidence of adverse side-effects. The primary consequence of in vivo administration of MPA/MMF is on T and B cell depletion, but T and B cells that arise from the engrafted hematopoietic stem cells (HSCs) that are transduced with the dual promoter lentiviral vector are resistant to MPA and have a selective growth advantage. Treatment with low doses of MMF increases the number of therapeutic lymphocytes in vivo, without affecting the original engraftment, with minimal toxicity. This, in turn, increases the total number of circulating cells that are expressing the transgene of interest, and thus the cells are able to express and/or secrete the protein encoded by the transgene, for example, secreting α-gal A, GBA protein, or other protein expressed by other transgenes of interest, which can lead to treatment or correction of the disease associated with the transgene, e.g., the lysosomal storage disorder.

This platform technology is broadly applicable to hematopoietic cell-based treatment of many inherited disorders, including other lysosomal storage disorders or hematopoietic system diseases in which a selective growth advantage of altered hematopoietic cells allows for treatment of the disease.

In one aspect, a dual promoter lentivirus vector that expresses α-galactosidase A and IMPDH2(IY) when transduced into a host cell and confers resistance to mycophenolic acid (MPA) and/or mycophenolate mofetil (MMF) in vitro and/or in vivo is provided herein.

In another aspect, the invention provides a dual promoter lentivirus vector that expresses a protein of interest encoded by a transgene along with IMPDH2(IY) when transduced into a host cell and confers resistance to mycophenolic acid (MPA) and/or mycophenolate mofetil (MMF) in vitro and/or in vivo. Suitable, the transgene can encode for a protein or enzyme deficient or dysregulated in a disease or disorder.

In another aspect, the invention provides a method of treating a subject in need of treatment for Fabry disease. The method comprises the steps of i) obtaining hematopoietic stem cells (HSCs) from the patient and/or HSCs from a suitable donor, ii) transducing the HSCs ex vivo or in vitro with a dual promoter lentivirus vector that expresses α-gal A and IMPDH2(IY) in the transduced cells, iii) introducing the transduced HSCs into the subject, and iv) administering to the subject an amount of mycophenolate mofetil (MMF) sufficient to enrich the population of lentivirus vector transduced hematopoietic cells, e.g. T and B cells in the subject.

In another aspect, a method of treating a subject having a disease associated with a defect in a single protein are provided. The method comprising the steps of i) obtaining hematopoietic stem cells (HSCs) from the patient and/or HSCs from a suitable donor, ii) transducing the HSCs ex vivo or in vitro with a dual promoter lentivirus vector that expresses a functional form of the protein of interest encoded by a transgene, and IMPDH2(IY) in the transduced cells, iii) introducing the transduced HSCs into the subject, and iv) administering to the subject an amount of mycophenolate mofetil (MMF) sufficient to enrich for the population of hematopoietic cells (e.g. T cells and B cells) that are formed from the lentivirus vector transduced HSCs engrafted in the subject.

In a particular aspect, the disease is Gaucher disease and the single protein is beta-glucocerebrosidase (GBA protein) encoded by the GBA transgene.

In another particular aspect, the disease is Fabry disease and the single protein is alpha galactosidase. In one aspect, the dual promoter is SEQ ID NO:3 or a sequence that has at least 80% identity to SEQ ID NO:3.

In another aspect, the dual promoter lentivirus vector expresses GBA protein and IMPDH2(IY). A suitable vector is found in SEQ ID NO:4 or a sequence that has at least 80% identity to SEQ ID NO:4, alternatively a sequence that has at least 90% identity, alternatively a sequence that has about 95% identity, alternatively a sequence that has at least 98% identity, alternatively a sequence that has about 99% sequence identity.

In another aspect, the invention provides a method of treating a subject in need of treatment for Gaucher disease. The method comprises the steps of i) obtaining hematopoietic stem cells (HSCs) from the patient and/or HSCs from a suitable donor, ii) transducing the HSCs ex vivo or in vitro with a dual promoter lentivirus vector that expresses GBA protein and IMPDH2(IY) in the transduced cells, iii) introducing the transduced HSCs into the subject, and iv) administering to the subject an amount of mycophenolate mofetil (MMF) sufficient to enrich the population of lentivirus vector transduced hematopoietic cells, e.g. T and B cells in the subject. The method is able to treat or reduce one or more symptoms of Gaucher disease.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing depicting exemplary lentiviral vectors of the present invention.

FIG. 2A is a plasmid map of the dual promoter lentiviral vector with two multiple cloning sites (MCS) for cloning of a transgene and a resistance gene.

FIG. 2B is a plasmid map of the dual promoter lentiviral vector containing the mutant IMPDH2 gene (IMPDH2(IY)) and a multiple cloning site in which a transgene can be cloned and expressed. FIG. 2C is the plasmid map of the dual promoter lentiviral vector containing the mutant IMPDH2 gene (IMPDH2(IY)) and AGA codon optimized transgene.

FIG. 3 is a schematic representation of methods of producing lentivirus encoding the transgene and IMPDH2(IY).

FIG. 4A depicts the schematic for the experiment used for testing the enrichment of cells transduced by the lentivirus in vitro using Jurkat cells.

FIG. 4B depicts the results of live cell count for the different Jurkat cell populations.

FIG. 4C depicts the results for vector copy number per genome for the different Jurkat cell populations.

FIG. 4D depicts the live vector copies calculated for cells on day 16.

FIG. 4E depicts α-galactosidase A (α-gal A) activity in the cell lysates.

FIG. 4F depicts α-gal A activity in the supernatants.

FIG. 5 is a schematic showing how IMPDH2(IY) transduced cells can be selectively enriched by treatment with MMF in vivo.

FIG. 6A is a schematic depicting the engraftment procedure for experiments to test enrichment in vivo in a mouse model.

FIG. 6B shows the results of mice engrafted with HSPCs transduced with the dual promoter lentiviral vector after being fed chow containing MMF for 0-60 days.

FIG. 7 is a schematic representation of the method of treating a disease using the hematopoietic stem cells transduced with the lentiviral vector of the present invention for Fabry disease.

FIG. 8 shows the chemical MMF and its converted form of MPA.

FIG. 9 is a schematic showing the pathway in which MMF works within cells and how the mutant form of IMPDH2 interacts with this pathway.

FIG. 10 is a schematic showing other exemplary dual promoter lentiviral vectors expressing GBA protein used in Examples.

FIG. 11 is a vector map of the dual promoter lentiviral vector that expresses GBA protein and IMPDH2(IY) (SEQ ID NO:4) for use in the treatment of Gaucher disease.

FIG. 12 is a representative graph showing the increase in GBA enzyme activity in culture supernatant of cells transduced with dilutions of LV using the GBA protein expressing lentiviral vectors in FIG. 10.

FIG. 13 shows representative experiments demonstrating increased enzyme activity in cell culture supernatant in cells transduced with the GBA+(IY) virus and treated with MPA.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The present invention provides dual promoter lentiviral vectors that can be used for the treatment of diseases and disorders that are the result of the dysregulation or dysfunction of a single protein or enzyme. Specifically, the present invention provides a dual promoter lentiviral vector that expresses a transgene of interest and a mutant form of inosine-5′-monophosphate dehydrogenase 2 (IMPDH2) that contains T333I and S351Y (IY) mutations within the gene (IMPDH2(IY)) which confers resistance to MPA in a trans-dominant manner. The vector confers resistance to mycophenolic acid (MPA) and/or mycophenolate mofetil (MMF) in vitro and in vivo when expressed within the host cell. As such the dual promoter lentiviral vectors may be used to transform hematopoietic stem cells that confers mycophenolic acid (MPA) resistance to transduced hematopoietic cells, including the derived B-and T-cells in the white cell population. MPA, which is delivered in vivo as the FDA approved drug mycophenolate mofetil (MMF), inhibits an enzyme needed for the growth of T cells and B cells. As discussed above, the primary consequence of in vivo administration of MPA/MMF is on T and B cell depletion, but in addition, the present invention shows that T and B cells that arise from the engrafted hematopoietic stem cells (HSCs) that are transduced with the dual promoter lentiviral vector are resistant to MPA and have a selective growth advantage. Treatment with low doses of MMF increases the number of therapeutic transduced lymphocytes in vivo with minimal toxicity. This, in turn, increases the total number of circulating cells that are expressing the transgene of interest, and thus the cells are able to express and/or secrete the protein encoded by the transgene of interest, which can lead to treatment or correction of the disease associated with the transgene, e.g., the lysosomal storage disorder.

This platform technology is broadly applicable to hematopoietic cell-based treatment of many inherited disorders, including other lysosomal storage disorders or hematopoietic system diseases in which a selective growth advantage of altered T cells or B cells allows for treatment of the disease. In some embodiments, this technology is applicable to hematopoeiteic cell-based treatment of inherited disorders, for example, clotting disorders, in which a

The transgene may be any protein or enzyme that is associated with a disease or disorder. In some instances, the transgene is an enzyme or protein associated with a lysosomal storage disorder. In one embodiment, the lysosomal storage disorder is Fabry disease and the transgene is AGA which encodes the enzyme α-galactosidase A (α-gal A). As used herein, the term α-galactosidase A and α-gal A are used interchangeably to refer to α-galactosidase enzyme (protein). In suitable embodiments, the transgene is a human protein, for example, α-galactosidase A, for treatment of Fabry disease. Suitable transgenes include transgenes that are not immunogenic (e.g. are preferably from human), and/or (b) have no gross effect on the hematopoietic system. Another suitable embodiment, the transgene encodes for a human protein for beta-glucocerebrosidase or glucosylceramidase (GBA protein or GCase). In suitable embodiments, the vector encodes for and expresses a transgene lacking in the disease, particularly lysosomal storage diseases, for example, but not limited to, α-gal A for Fabry disease, beta-glucocerebrosidase (GBA) for Gaucher disease, acid ceramidase (ASAH1) for Farber disease, acid α-glucosidase (GAA, also known as acid maltase) for Pompe disease.

Suitable diseases and transgenes may relate to lysosomal storage disorders, including inherited metabolic disorders that result from defects in lysosomal function. Lysosomal storage disorders are caused by lysosomal dysfunction usually as a consequence of deficiency of a single enzyme required for the metabolism of lipids, glycoproteins (sugar containing proteins) or so-called mucopolysaccharides. Suitable lysosomal storage disorders and transgenes that are contemplated to be treated by this method include, but are not limited to, for example, mucolipidosis type II and IIIa (N-acetylglucosamine-1-phosphate transferase), galactosialidosis (cathepsinA), GM2-AP deficiency (GM2-AP), Niemann-Pick disease type C2 (NPC2), Niemann-Pick disease type C1 (NPC1), Salla disease (sialin), Krabbe disease (galactosylceramidase), GM1 gangliosidosis (beta-galactosidase), GM gangliosidoses (beta-hexosaminidases), Fabry disease (α-galactosidase A), Schindler disease (α-galactosidase B), Sandhoff disease (beta-hexosaminidase B), Tay-Sachs disease (beta-hexosaminidase A), Gaucher's disease (glucocerebrosidase), lysosomal acid lipase deficiency (lysosomal acid lipase (LAL) enzyme), sulfatidosis (sulfatase enzymes), Metachromatic leukodystrophy (MLD, also called arylsulfatase A deficiency, enzyme arylsulfatase A (ARSA)), multiple sulfatase deficiency disease or Austin (formylglycine-generating enzyme), mucopolysaccharidoses, Hurler syndrome (α-L iduronidase), Scheie and Hurler-Scheie syndrome (α-L iduronidase), Hunter syndrome (iduronate-2-sulfatase (I2S)), Sanfilippo syndrome (heparan N-sulfatase (MPS-IIIA)), N-acetyl-α-D-glucosaminidase (MPS-IIIB), acetyl-CoA: α-glucosaminidine acetyltransferase (MPS-III C), N-acetylglucosamine-G-sulfate sulfatase (MPS-IIID), Maroteaux-Lamy syndrome (arylsulfatase B), Sly syndrome (beta-glucuronidase), hyaluronidase deficiency (hyaluronidase), Sialidosis (α-N-acetyl neuraminidase (sialidase)), Inclusion-cell (I-cell) disease (phosphotransferase), Mucolipidin 1 deficiency (mucolipidin 1), neuronal ceroid lipofuscinosis (CLN) family (palmitoyl-protein thioesterase 1 (PPT-1), tripeptidyl peptidase I (TPP1), battenin (CLN3), Ceroid-lipofuscinosis neuronal protein 6 (CLN6 gene), Ceroid-lipofuscinosis neuronal protein 5, Ceroid-lipofuscinosis neuronal protein 8, Major facilitator superfamily domain containing 8, cathepsin D, among others), α-mannosidosis (α-mannosidase enzyme), beta-mannosidosis (beta-mannosidase), Aspartylglucosaminuria (AGU) (aspartylglucosaminidase), fucosidosis (α-L-fucosidase enzyme), cystinosis (cystinosin), pycnodysostosis (enzyme cathepsin K), infantile free sialic acid storage disease (solute carrier family 17), among other lysosomal storage disorders known in the art (protein/enzymes are within the brackets after the disease).

In one embodiment, the present invention employs a dual promoter vector that expresses α-galactosidase A (α-gal A) (for example, SEQ ID NO:5) and (IMPDH2(IY)) (SEQ ID NO: 7). A suitable nucleic acid sequence encoding α-gal A includes, for example, but not limited to, SEQ ID NO:7 or a sequence that is about 80% sequence identity to SEQ ID NO:7, alternatively at least 85% identity to SEQ ID NO:7, alternatively at least 90% identity to SEQ ID NO: 7, alternatively at least 95% sequence identity to SEQ ID NO:7, alternatively at least 98% identity to SEQ ID NO:7, alternatively at least 99% identity to SEQ ID NO:7. A suitable vector includes, for example, but not limited to, SEQ ID NO: 3 or a vector that is about 80% sequence identity to SEQ ID NO:3, alternatively at least 85% identity to SEQ ID NO:3, alternatively at least 90% identity to SEQ ID NO:3, alternatively at least 95% sequence identity to SEQ ID NO:3, alternatively at least 98% identity to SEQ ID NO:3.

In another embodiment, the present invention employs a dual promoter vector that expresses glucocerebrosidase (or beta-glucocerebrosidase, for example encoded by the transgene of SEQ ID NO:6) and IMPDH2(IY) (SEQ ID NO:7). A suitable nucleic acid sequence encoding beta-glucocerebrosidase includes, for example, but not limited to, SEQ ID NO: 6 or a sequence that is about 80% sequence identity to SEQ ID NO:6, alternatively at least 85% identity to SEQ ID NO:6, alternatively at least 90% identity to SEQ ID NO:6, alternatively at least 95% sequence identity to SEQ ID NO:6, alternatively at least 98% identity to SEQ ID NO:6, alternatively at least 99% identity to SEQ ID NO:6. A suitable vector includes, for example, but not limited to, SEQ ID NO:4 or a vector that is about 80% sequence identity to SEQ ID NO:4, alternatively at least 85% identity to SEQ ID NO:4, alternatively at least 90% identity to SEQ ID NO:4, alternatively at least 95% sequence identity to SEQ ID NO: 4, alternatively at least 98% identity to SEQ ID NO:4.

In another embodiment, the present invention employs a dual promoter vector that expresses acid ceramidase (ASAHI, for example encoded by the transgene of SEQ ID NO: 8) and IMPDH2(IY) (SEQ ID NO:7). This vector may be used to transduce HSCs for treatment of Faber disease. A suitable nucleic acid sequence encoding ASAHI includes, for example, but not limited to, SEQ ID NO:8 or a sequence that is about 80% sequence identity to SEQ ID NO:8, alternatively at least 85% identity to SEQ ID NO:8, alternatively at least 90% identity to SEQ ID NO:8, alternatively at least 95% sequence identity to SEQ ID NO:8, alternatively at least 98% identity to SEQ ID NO:8, alternatively at least 99% identity to SEQ ID NO: 8.

In another embodiment, the present invention employs a dual promoter vector that expresses GAA, for example encoded by the transgene of SEQ ID NO:9 and IMPDH2(IY) (SEQ ID NO:7). This vector may be used for transducing HSCs for treatment of Pompe disease. A suitable nucleic acid sequence encoding GAA includes, for example, but not limited to, SEQ ID NO:9 or a sequence that is about 80% sequence identity to SEQ ID NO:9,alternatively at least 85% identity to SEQ ID NO:9, alternatively at least 90% identity to SEQ ID NO: 9, alternatively at least 95% sequence identity to SEQ ID NO:9, alternatively at least 98% identity to SEQ ID NO:9, alternatively at least 99% identity to SEQ ID NO:9.

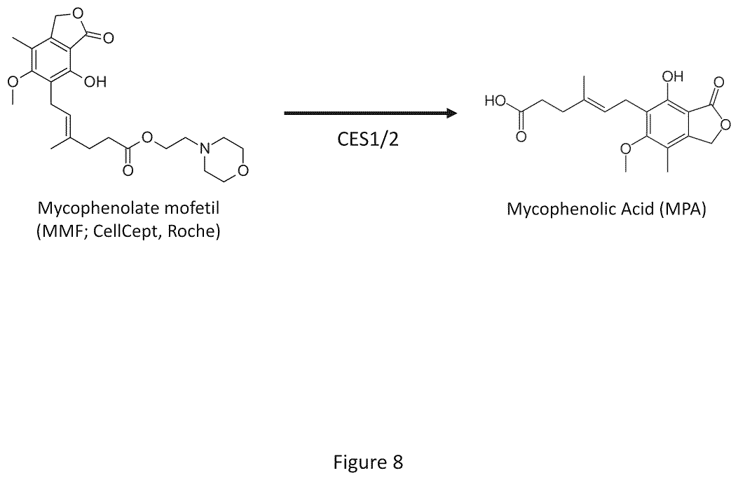

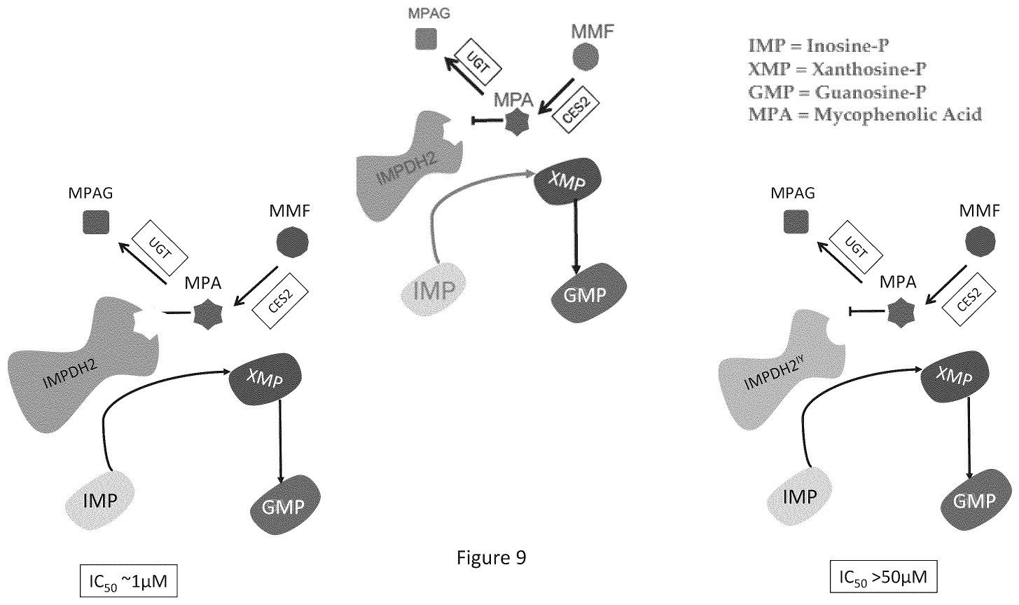

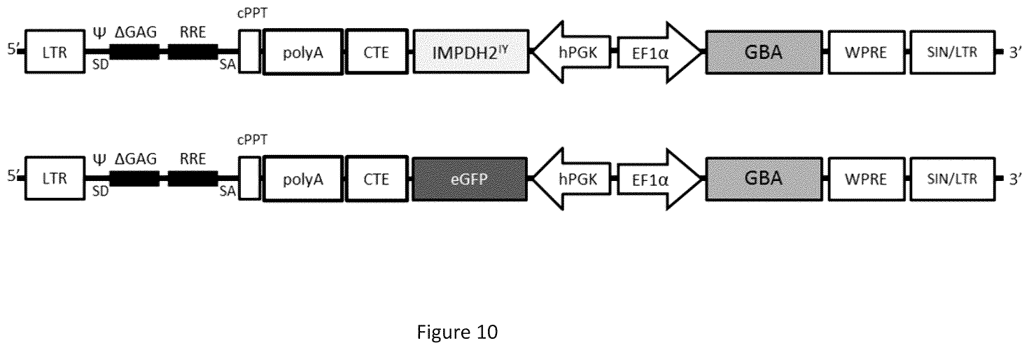

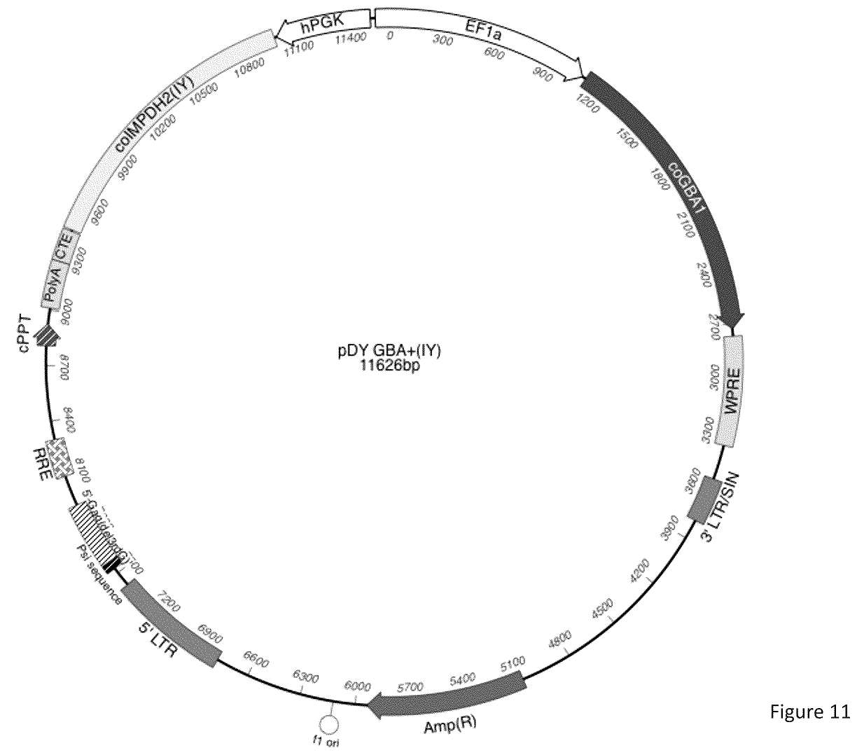

IMPDH2(IY) is resistant to mycophenolic acid (MPA). Mycophenolate Mofetil (MMF) is the pro-drug for MPA and is used routinely in the clinic for a variety of indications, with low incidence of adverse side-effects. FIG. 8 shows the chemical structures of MMF and MPA and FIG. 9 shows the pathway that MMF and/or MPA disrupts through inhibition of IMPDH2(IY), that is, the MPA mechanism of action. MMF is a widely used and well-studied immunosuppressant given by oral administration, it is activated and absorbed by the gut epithelium and also by the liver. MMF has been shown to have minimal occurrence of adverse events at low doses. MMF targets activated lymphocytes and prevents activation induced proliferation of T cells and also prevents maturation of B cells. There is also some evidence that MMF inhibits cytotoxicity of NK cells. MPA is a reversible, non-competitive inhibitor of inosine-5′-monophosphate dehydrogenase (IMPDH). IMPDH is the rate-limiting enzyme in de novo GMP synthesis. T and B cells are highly dependent on IMPDH2, while other tissues can utilize a salvage pathway. Thus, a mutant form of IMPDH2, which has two mutations (T333I and S351Y (IY)) confer resistance to MPA (and thus MMF) in a trans-dominant manner.

The primary consequence of MPA administration is T and B cell depletion. By expressing IMPDH2(IY), T and B cells that arise from transduced and engrafted hematopoietic stem cells (HSCs) are resistant to MPA. Treatment with low doses of MMF can increase the number of therapeutic lymphocytes, without affecting the original engraftment, while causing minimal toxicity. This, in turn, increases the total number of circulating cells that are expressing and secreting the transgene, for example, α-galactosidase A, which can lead to better correction of the disease. Previous attempts at amplification of therapeutic cells have targeted the original HSC engraftment. These strategies either provide HSCs with resistance to cytotoxic agents, or with drug inducible growth modules. Administration of cytotoxic drugs is often associated with severe adverse events, and repeat-administration is also not ideal. Drugs used for inducible growth are not approved for use in patients, limiting their potential. High induction of growth in HSCs can also be associated with stem cell exhaustion, limiting the utility of the graft. Targeting the HSC compartment with a chemical compound is also difficult, due to their unique niche. The aforementioned concerns are ameliorated with the use of our enrichment strategy described herein, since it excludes the HSC compartment.

A suitable nucleic acid sequence encoding for IMPDH2(IY) is found in SEQ ID NO: 7. Suitably, the dual lentiviral vectors comprise SEQ ID NO:7 or a sequence that is about 80% sequence identity to SEQ ID NO:7, alternatively at least 85% identity to SEQ ID NO:7, alternatively at least 90% identity to SEQ ID NO:7, alternatively at least 95% sequence identity to SEQ ID NO:7, alternatively at least 98% identity to SEQ ID NO:7, alternatively at least 99% identity to SEQ ID NO:7. Suitable nucleic acid sequence allow for the expression of IMPDH2(IY) which confers resistance to MPA.

The Examples demonstrate the enrichment of lentiviral-transduced cells in vitro and that this enrichment leads to increased enzyme activity and secretion of enzyme from the cells. Further, we show that this enrichment can be carried out in vivo by administering MMF to the subject.

Current gene therapy involving hematopoietic cells targets correction of cells and engraftment into hosts. The cells are then left on their own to engraft and cure the host. Yet oftentimes the transduction or engraftment efficiency is low. The current method gives a way to enrich for transduced cells in vivo and allows some gating as to how selective and strong that enrichment is depending on the administration of the MMF. The present methods also allow for cells harboring this lentiviral vector to be enriched for even years down the road to renew the correcting cell population expressing the transgene of interest.

In some instances, the dual promoters are promoters known in the art. In one embodiment, the first promoter is hPGK promoter and the second promoter is EF1α promoter. Other promoters are able to be used in the practice of the present invention, as long as the promoters allow for expression of the downstream gene.

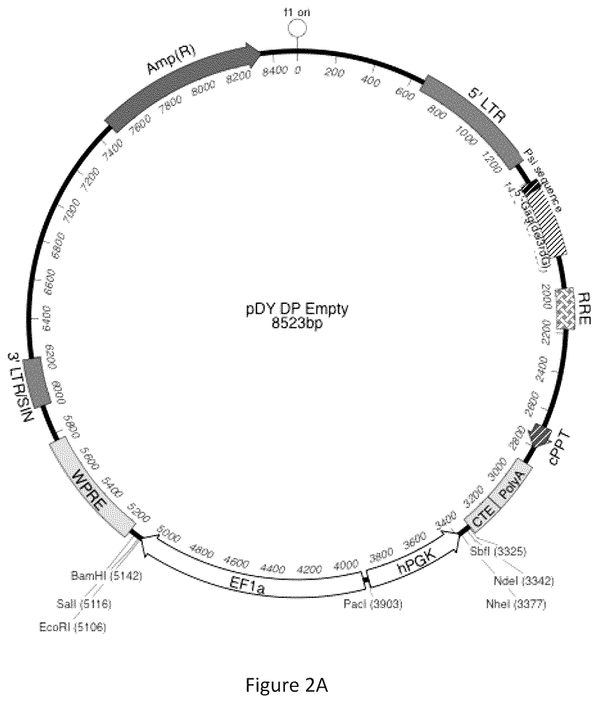

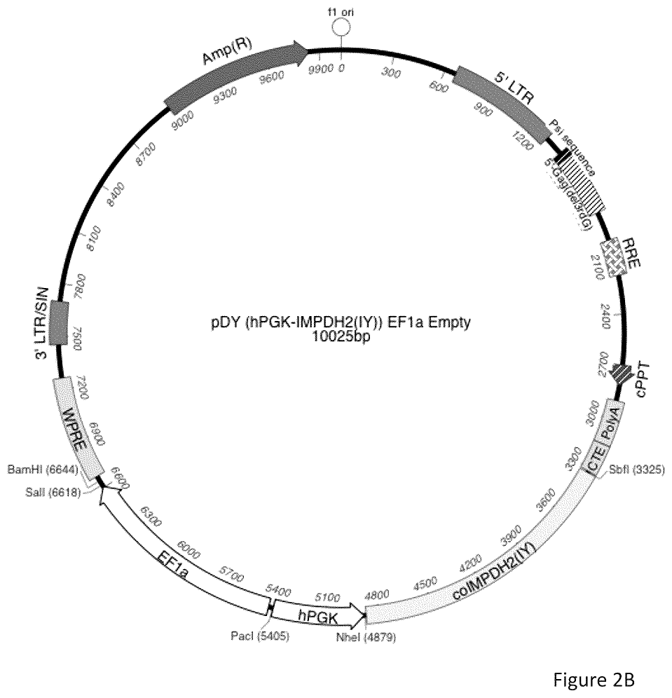

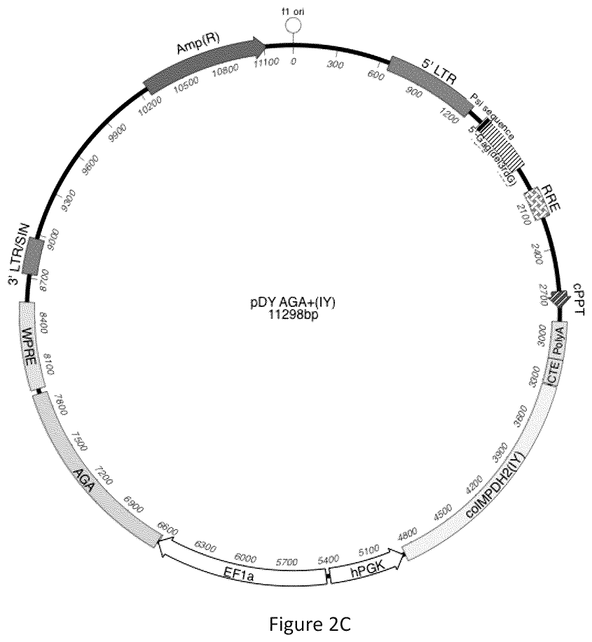

In another embodiment, a dual promoter lentiviral vector that expresses α-galactosidase A (encodes the AGA transgene) and IMPDH2(IY) when transduced into a host cell and as such confers resistance to mycophenolic acid (MPA) and/or mycophenolate mofetil (MMF) in vitro and/or in vivo is provided. A suitable dual promoter lentivirus vector that expresses α-galactosidase A and IMPDH2(IY) is depicted in FIG. 2B and the sequence is provided in SEQ ID NO: 3.

In another embodiment, a dual promoter lentiviral vector that expresses a transgene and IMPDH2(IY) when transduced into a host cell is provided. The vector confers resistance to mycophenolic acid (MPA) and/or mycophenolate mofetil (MMF) in vitro and/or in vivo in the host cell. A suitable dual promoter lentivirus vector contains a multiple cloning site to clone the transgene of interest and IMPDH2(IY) is provided in SEQ ID NO: 2.

In some embodiments, the vector comprises the SEQ ID NO:2 wherein the target gene of interest is cloned into the multiple cloning site. Suitable target gene of interests able to be cloned into the multiple cloning site of SEQ ID NO:2 include, but are not limited to, for example, AGA (SEQ ID NO:5), GBA (SEQ ID NO:6), ASAHI (SEQ ID NO:7) and GAA (SEQ ID NO:9). One skilled in the art would be able to encode the target gene of interest into the vector of SEQ ID NO:2 without undue experimentation.

Methods of treating a patient using the dual promoter lentiviral vector described herein are provided. The method comprises, i) obtaining hematopoietic stem cells (HSCs) from the patient and/or HSCs from a suitable donor, ii) transducing the HSCs ex vivo with a dual promoter lentivirus vector containing IMPDH2(IY) and a transgene (e.g. AGA transgene), iii) introducing the transduced HSCs into the subject, and iv) administering to the subject an amount of mycophenolate mofetil (MMF) sufficient to enrich the population of lentivirus vector transduced hematopoietic cells, for example, T and B cells in the subject.

One embodiment provides a method of treating a subject having a disease associated with a defect in a single protein or enzyme, the method comprising the steps of i) obtaining hematopoietic stem cells (HSCs) from the patient and/or HSCs from a suitable donor, ii) transducing the HSCs ex vivo with a dual promoter lentivirus vector that expresses a suitable form of the protein or enzyme and IMPDH2(IY) in the transduced cells, iii) introducing the transduced HSCs into the subject, and iv) administering to the subject an amount of mycophenolate mofetil (MMF) sufficient to enrich the population hematopoietic cells arising from lentivirus vector transduced HSCs engrafted in the subject.

In some embodiments, the subject has a lysosomal storage disorder (LSD), and the transgene encodes an enzyme or protein associated with the LSD that is deficient in the subject. In some embodiments, the LSD is Fabry disease and the transgene is AGA, the LSD is Gaucher disease and the transgene of interest is GBA, the LSD is Faber disease and the transgene is ASAH1, or the LSD is Pompe and the transgene is GAA.

Another embodiment provides a method of treating Fabry disease patients by:

1) obtaining HSCs from the patient or a suitable donor;

2) transducing the HSCs ex vivo with a dual promoter lentivirus vector that expresses α-gal A and IMPDH2(IY) in the transduced cells;

3) introducing the transduced HSCs and/or hematopoietic cells back into the patient; and,

4) administering mycophenolate mofetil (MMF) to the patient in order to enrich the population of lentivirus vector transduced T and B cells engrafted in the patient, thereby increasing the amount of α-gal A produced in the body. The method treats one or more symptoms of Fabry disease.

In some embodiments, the lysosomal storage disorder is Farber disease (also known as Farber's lipogranulomatosis, ceramidase deficiency, “Fibrocytic dysmucopolysaccharidosis,” and “Lipogranulomatosis”) and the transgene expresses ASAH1. Farber disease is an extremely rare autosomal recessive lysosomal storage disorder marked by a deficiency in the enzyme acid ceramidase that causes an accumulation of a waxy class of lipids known as sphingolipids, in particular ceramide, leading to abnormalities in the joints, liver, throat, visceral tissues and central nervous system. Suitable embodiments provide T-Rapa cells expressing N-Acylsphingosine Amidohydrolase 1 (ASAH1) for the treatment of Farber disease. As used in the present invention, a suitable dual promoter lentiviral vector expresses ASAHI and IMPDH2(IY) in the HSC cells. For example, a suitable vector can express ASAHI using the ASAHI transgene of SEQ ID NO:8 or a sequence having 80% similarity to SEQ ID NO:8. Suitably, the ASAHI transgene will have at least 80% similarity to the SEQ ID NO:8, alternatively at least 85% sequence similarity to SEQ ID NO:8, alternatively at least 90% sequence similarity to SEQ ID NO:8, alternatively at least 95% sequence similarity to SEQ ID NO:8, alternatively at least 98% sequence similarity to SEQ ID NO: 8, alternatively at least 99% sequence similarity to SEQ ID NO:8, alternatively at least 100% sequence similarity to SEQ ID NO:8.

Pompe disease is an inherited disorder resulting from the buildup of a complex sugar called glycogen in the body's cells resulting in accumulation of glycogen in certain organs and tissues, especially muscles, which impairs their ability to function normally. Mutations within the GAA gene cause Pompe disease as the GAA gene provides instructions for producing an enzyme called acid α-glucosidase (also known as acid maltase). This enzyme is active in lysosomes which serve as recycling centers within cells. The enzyme normally breaks down glycogen in lysosomes into a simpler sugar called glucose, which is the main energy source for most cells. In some embodiments, T-Rapa cells expressing GAA are used to treat a subject having Pompe disease. As described above, dual lentiviral vectors expressing GAA and IMPDH2(IY) transgene within transduced HSCs. In one embodiment, the dual promoter vectors, can express the GAA transgene of SEQ ID NO:9 or a sequence at least 80% similar to SEQ ID NO:9. Suitably, the GAA transgene will have at least 80% similarity to the SEQ ID NO: 9, alternatively at least 85% sequence similarity to SEQ ID NO:9, alternatively at least 90% sequence similarity to SEQ ID NO:9, alternatively at least 95% sequence similarity to SEQ ID NO:9, alternatively at least 98% sequence similarity to SEQ ID NO:9, alternatively at least 99% sequence similarity to SEQ ID NO:9, alternatively at least 100% sequence similarity to SEQ ID NO:9.

“Treat” or “treating” includes reducing, inhibiting, or eliminating one or more symptom of the disease or management and care of a subject for the purpose of combating the disease. Treating includes the administration of the HSCs of present invention to prevent or reduce the onset of the symptoms or complications, alleviating the symptoms or complications, or eliminating the disease, condition, or disorder. The aim of treatment includes the alleviation or prevention of symptoms, slowing or stopping the progression or worsening of a disease, disorder, or condition and/or the remission of the disease, disorder or condition. In certain embodiments, the treatment comprises HSCs transduced with the dual promoter and treatment with MMF. The term “treatment” can be characterized by at least one of the following: reducing, ameliorating, slowing or inhibiting one or more symptom of the disease or disorder. In some embodiments, the optimum effective amount can be readily determined by one skilled in the art using routine experimentation.

Symptoms of Fabry disease are known in the art and include, but are not limited to, pain or burning in the hands and/or feet, rash, narrowing of blood vessels, cloudy vision, hearing loss, ringing in the ears, sweating less than normal, stomach pain, kidney problems, high blood pressure, heart failure, enlarged heart, osteoporosis, among others.

In another embodiment, the invention provides a method of treating Gaucher disease by: 1) obtaining HSCs from the patient or a suitable donor; 2) transducing the HSCs ex vivo with a dual promoter lentivirus vector that expresses GBA protein and IMPDH2(IY) in the transduced cells (for example the vector of SEQ ID NO:4); 3) introducing the transduced HSCs and/or hematopoietic cells back into the patient; and, 4) administering mycophenolate mofetil (MMF) to the patient in order to enrich the population of lentivirus vector transduced T and B cells engrafted in the patient, thereby increasing the amount of GBA protein/enzyme produced in the body. The method treats one or more symptoms of Gaucher disease.

Symptoms of Gaucher Disease include, for example, an abnormally enlarged liver and/or spleen (hepatosplenomegaly), low levels of circulating red blood cells (anemia), low levels of platelets (thrombocytopenia), and skeletal abnormalities, among others.

The term “subject” or “patient” are used interchangeably and refer to a mammalian subject, for example, a mouse, a rat, a monkey, a human, etc. In a preferred embodiment, the subject is a human. It is contemplated that the subject or patient may have already been treated with one or more therapies for the lysosomal storage disorder before undergoing the treatment contemplated herein.

The host cell is suitably a hematopoietic cell, for example hematopoietic stem cells (HSCs).

In suitable embodiments, the MMF is administered at an effective dosage. An “effective dosage” refers to a dosage that allows for selective enrichment for T and B cells that express the transgene via the lentiviral vector with minimal side effects. In one embodiment, the effective dosage is a low dosage. Suitable low dosages include, but are not limited to, for example, 0.1-5 mg/kg body weight given TIB (three times a day), alternatively include from about 0.1-3 mg/kg body weight given TIB. Alternatively, the effective dose may include higher doses of MMF. Suitable higher dosage of MMF for practice of this invention include MMF in an amount of about 5-10 mg/kg body weight TID (three times a day), alternatively 1000 mg given BID (two times a day). Suitably, an “effective amount” of MMF will result in a blood concentration within the subject of about 0.4 to about 2 μM free mycophenolic acid (MPA). Suitable dosages to obtain this blood concentration are readily determined by a physician treating the subject. MMF may also be substituted for MPA formulations (Myfortic, Novartis, or approved generic).

Methods of obtaining HSCs from a subject are known in the art. For example an agent may be used to stimulate mobilization of HSCs to the peripheral blood. Suitable agents are known in the art and include, but are not limited to, for example, G-CSF or plerixafor (Mozobil, Sanofi).

HSCs can be transduced ex vivo using a suitable amount of lentivirus, for example, using a MOI (multiplicity of infection) of 1-100, preferably a MOI of 1-30, alternatively a MOI of 1-20, alternatively a MOI of 1-10. The cells may be exposed to the lentivirus for from 10-24 hours, suitably for example for 12 hours. Cytokines may be added to the culture medium during transduction. After transduction, the cells can be either transferred back into the patient or cryopreserved for later transplantation, or a combination of both. In some instances, the transduced cells may be cultured for a number of days before being transferred or cryopreserved. Suitable methods of cryopreservation are known in the art.

Suitable methods of cryopreservation include, but are not limited to, suspending the cells in a cryopreservation medium and storing the cells at −80° C. to −196° C., preferably below −80° C. Suitable cryopreservation media are known in the art and may comprise some combination of base medium, cryopreservative (e.g., DMSO) and a protein source. For example, a suitable cryopreservation medium may comprise complete medium and 10% glycerol, complete medium containing 10% DMSO (dimethlysulfoxide), or 45% cell-conditioned medium with 45% fresh medium and 10% glycerol or DMSO. In alternative embodiments, the cryopreservation medium may be serum free, for example, comprises 46.25% cell-conditioned serum-free medium with 46.25% fresh serum-free medium and 7.5% DMSO.

Subjects may be conditioned before transfer of the hematopoietic cells. Suitable methods of conditioning the patient are known in the art, and include, for example, low-dose regimen (e.g. 30-50% of normal dose) of melphalan or another chemotherapeutic drug. For example, a patient may be treated for 1-3 days by administration once a day with melphalan, as determined by a physician. One skilled in the art of treating the subject will be able to provide an appropriate conditioning regimen. The purpose of conditioning is to reduce the number of endogenous bone marrow cells to provide an advantage to the newly transferred transduced HSCs to engraft and grow.

After conditioning, the patients may receive at least one administration of transduced cells, for example, from about 2-10 ×10⁶ transduced cells/kg body weight intravenously. Suitably amounts of cells include, but are not limited to, e.g. 2×10⁶ transduced cells/kg, 2.5×10⁶ transduced cells/kg, 3×10⁶ transduced cells/kg, 4×10⁶ transduced cells/kg, 4.5×10⁶ transduced cells/kg, 5×10⁶ transduced cells/kg, and include amounts and ranges in between. In some embodiments, subjects may receive additional administration of cells after the first administration at later time points (e.g. hours, days, weeks or months after the first administration). In one embodiment, additional cells may be administered in the same week as the first treatment (e.g. day 2-7) or in the additional weeks post first administration (e.g. week 2-6). In some embodiments, the levels of transgene being expressed can be determined and if the levels become sufficiently low an additional conditioning and/or administration of transduced cells that were cryopreserved can be administered (e.g. weeks or months later).

Timing of MMF doses can be determined by one skilled in the art. In some embodiments, the MMF may be administered during conditioning regimen, depending on the source of the transferring cells. Suitable examples of MMF are described below and MMF can be administered continuously throughout the subject's lifetime or stopped for periods of time or when no longer necessary. In other embodiments, MMF is administered after transfer/engraftment of the transduced HSCs to the subject. Suitably, MMF may be administered for at least 0.5-2 years after engraftment. In some instances, MMF may be administered periodically over the subject's lifetime after transplantation to re-enrich for the transduced cells, e.g. may be administered 5 or 10 years after initial engraftments. MMF may be administered in low dosages continuously, or MMF may be administered non-continuously, for example, MMF may be administered after engraftment for a period of at least six months, followed by a period of time without MMF treatment (e.g. months or years), followed by a restarting of MMF treatment as a “booster” to re-enrich for transduced cells at later time points in the subject's life. Monitoring of transgene protein (e.g. α-gal A) levels within the subject may determine the length of time of administration and dose of MMF, or if additional booster treatment with MMF are needed at later time points after MMF treatment was stopped to re-enrich for more transduced cells to increase the systemic levels of the transgene (e.g. AGA transgene) expressed within the subject. In some embodiments, the transgene may reach suitable levels systemically within the subject where the subject may not need MMF treatment. However, if at later times the transgene systemic levels fall, then MMF treatment can be reinitiated to re-enrich for α-gal A expressing cells.

The methods contemplated herein can be adapted to other LSDs, because the dual promoter lentivirus vector can be modified to express the enzyme (transgene) that is deficient in other LSDs. In addition, there are other disorders, particularly hematopoietic disorders, that the method of the invention can be used to treat, specifically diseases that are related to a single gene defect. For example, hemaptopoeitic disorders that specifically relate to T or B cells may be treated by the methods described herein, including, but not limited to, for example, Wiskott-Aldrich Syndrome with the WAS protein, Leukodystrophies (X-linked and Metachromatic) (ABCD1 gene (adrenoleukodystrophy protein) and ARSA gene (arylsufatase A) protein, and the like. Other inherited diseases that are associated with a single gene defect include, but are not limited to, for example, X-linked agammaglbulinemia (BTK gene which encodes the BTK protein),

Specifically, in some embodiments, the methods can be used to treat hematopoietic disorders that are able to be treated with transduced hematopoietic stem cells that express a soluble protein encoded by a transgene of interest. For example, one type of suitable diseases are clotting factor deficiencies, for example, hemophilia A, a Factor VIII deficiency, hemophila B, a Factor IX deficiency, Von Willebrand's disease, a von Willebrand factor associated disease. A suitable transgene includes Factor VIII of SEQ ID NO:11, or a sequence that is at least 80% identity to SEQ ID NO:11, alternatively a sequence that has at least 85% identity, alternatively a sequence that has at least 90% identity, alternatively a sequence that has about 95% identity, alternatively a sequence that has at least 98% identity, alternatively a sequence that has about 99% sequence identity. A suitable vector encoding for Factor VIII is found in SEQ ID NO:12, or a sequence that is at least 80% identity to SEQ ID NO: 12, alternatively a sequence that has at least 85% identity, alternatively a sequence that has at least 90% identity, alternatively a sequence that has about 95% identity, alternatively a sequence that has at least 98% identity, alternatively a sequence that has about 99% sequence identity.

In addition, considering that HSCs have been used experimentally to treat a variety of non-hematopoietic diseases including spinal cord injuries, liver cirrhosis, and peripheral vascular disease, the inventors' dual promoter lentivirus vector and in vivo enrichment strategy can be used in the treatment of other diseases by enriching for transduced T and B cell populations that secrete therapeutic proteins targeting other diseases, for example, expression of therapeutic antibodies against tumor cells, or in the treatment of autoimmune diseases.

Current gene therapy involving hematopoietic cells targets correction of cells and engraftment into hosts. The cells are then left on their own to engraft and cure the host. However, the transduction or engraftment efficiency is often low. The current method provides a way to enrich for transduced cells in vivo and allows some degree of “gating” as to how selective and strong that enrichment is. In this regard, cells harboring the lentiviral vector could be enriched for even years down the road to renew the transduced cell population that express the desired transgene.

The invention overcomes the following problems associated with current HSC-based therapies:

Previous attempts at amplification of therapeutic cells have targeted the original HSC engraftment. Such inventions provide HSCs and their progeny with resistance to cytotoxic agents, or with drug inducible growth modules.

Administration of cytotoxic drugs is often associated with severe adverse events, and repeat-administration is also not ideal. MMF is used routinely in the clinic with a low incidence of adverse side-effects.

Drugs used for inducible growth are not approved for use in patients, limiting their potential. High induction of growth in HSCs can also be associated with stem cell exhaustion, limiting the utility of the graft. MMF is a commonly used, FDA approved drug that has no known effect on HSCs.

Targeting the HSC compartment with a chemical compound is also difficult, due to their unique niche. The enrichment strategy disclosed by the inventors would overcome this problem because it excludes the HSC compartment.

Many LSD patients may have been treated with standard of care, which is often enzyme replacement therapy. These therapies may lead to formation of an adaptive immune response against the therapeutic protein of interest, that is, the therapy is recognized as foreign material, for example against the enzyme replacement therapy. One such response is an adaptive immune response, for example formation of therapeutic-specific antibodies which occurs in most patients receiving enzyme replacement therapies, including, but not limited to, Fabry and Pompe disease, reduces the efficacy of the therapy dramatically and leads to clearance of the corrective protein. As such, patients that have pre-existing immune response against the corrective protein treated with gene therapy may have clearance of the therapeutic protein and therefore reduced efficacy. The inventors have observed this occurring in some patients that have been previously treated with enzyme therapy. The current methods described herein overcome the previous problems with standard enzyme replacement therapy in that the MMF used in the present methods not only provides a selective advantage to the transduced cells expressing the enzyme of interest, but also is able to prevent the proliferation of endogenous antibody producing B cells that may react with the therapeutic enzyme. The inventors therefore envision an additional benefit of their platform in that endogenous adaptive immune responses, for example B cells that produce antibodies against the therapeutic protein, can be suppressed in addition to enriching for vector positive B and T cells that should be tolerant and not react to the therapeutic protein. In this way, greater efficacy may be achieved especially in patients whose immune systems are not fully ablated.

Thus the methods of the present invention described herein may be used in patients that are fully ablated, partially ablated or have not undergone ablation prior to treatment with HSCs transduced with the dual promoter lentiviral vector.

“Percentage of sequence identity” or “sequence similarity” is determined by comparing two optimally aligned sequences over a comparison window, wherein the portion of the polynucleotide sequence in the comparison window may comprise substitutions, or additions or deletions (i.e., gaps) as compared to the reference sequence (which does not comprise substitutions, additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “similarity” of polynucleotide sequences means that a polynucleotide comprises a sequence that has at least 80% sequence identity. Suitable sequence similarity allows for small changes in the transgene that do not affect the function of the protein expressed by the transgene. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using programs such as BLAST using standard parameters. These values can be appropriately adjusted to determine corresponding identity of proteins encoded by two nucleotide sequences by taking into account codon degeneracy, amino acid similarity, reading frame positioning and the like.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The invention will be more fully understood upon consideration of the following non-limiting examples.

### EXAMPLES

### Example 1: Production of Dual Promoter Lentivirus Vectors

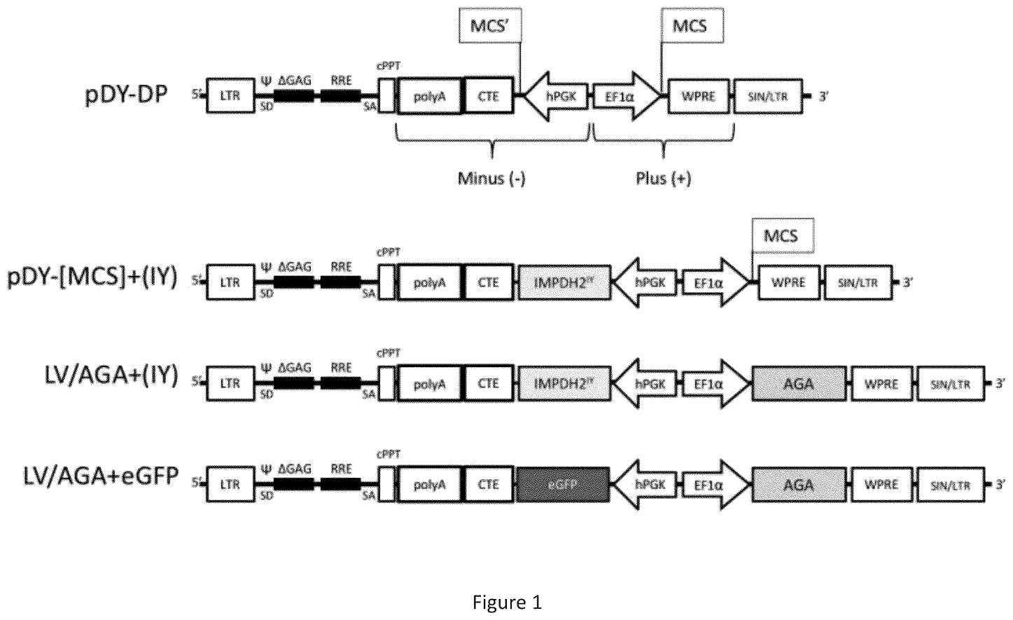

The present invention uses dual promoter lentivirus vectors to transfer a transgene and the resistance gene to mycophenolate mofetil (MMF) into the target cell. FIG. 1 shows the vector schematic for some suitable lentiviral vectors for use in the present invention. A dual promoter architecture (pDY-DP (SEQ ID NO:1) was designed and constructed using pDY as a backbone using standard molecular biology techniques. It is contemplated that right other dual promoter architectures and backbones may be used in the practice of the present invention without departing from the scope of the invention. Human-derived ubiquitous, constitutive promoters express transgenes of interest. For enrichment purposes, a vector with IMPDH2(IY) expressed from one promoter was constructed, with the ability to insert another transgene of interest from the other promoter (i.e. pDY−[MCS]+(IY), (SEQ ID NO:2)). A vector with AGA transgene was constructed for use in treating Fabry disease (SEQ ID NO:3) and a vector with GBA transgene was constructed for use in treating Gaucher disease (SEQ ID NO:4). The titer for these vectors are in the range of 1×10⁹ infectious viral particles (IVP)/mL. Vectors with enhanced green fluorescent protein (eGFP) instead of IMPDH2(IY) were used to measure expression and for use as a non-enrichment control. FIG. 2A-C and 11 show the vector maps of lentiviral vectors used in the present invention.

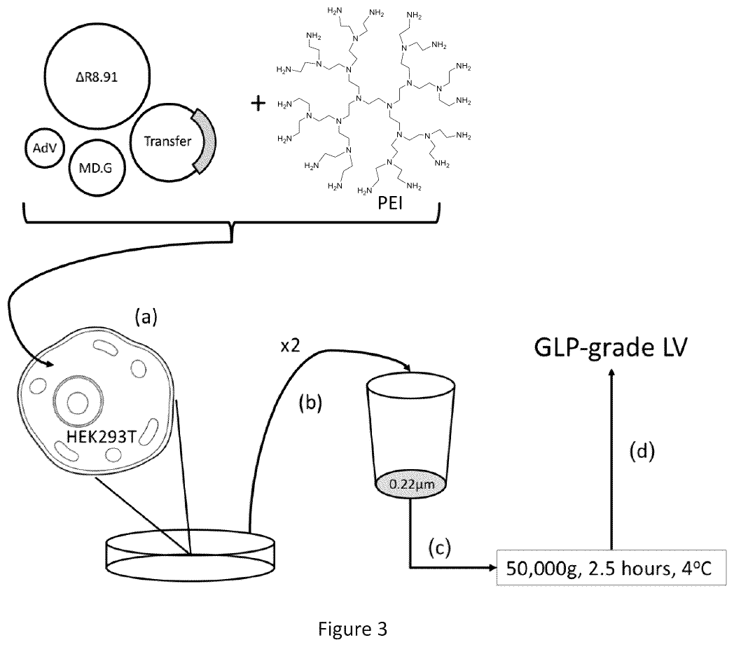

Suitable methods of producing lentiviral vectors are known in the art. A suitable protocol is shown in FIG. 3. Production of lentivirus includes the following steps: (a) Three packaging plasmids, pCMVΔR8.91 (Zufferey, et al. Nature Biotechnology 15: 871-874, 1997, incorporated by reference), pMD.G (Naldini et al., Science 272: 263-267, 1996, incorporated by reference) and pAdV (Promega, USA) are mixed in appropriate ratios with a plasmid encoding the transfer vector of interest. These are complexed with polyethylenimine (PEI) and transfected into HEK293T cells. Media is replaced after 16 hours. (b) Culture supernatant containing virus particles is harvested approximately 40 and 64 hours after transfection. The supernatant is filtered through 0.22 μm membrane filters to remove contaminants. (c) Collected supernatant is subjected to ultracentrifugation as indicated to concentrate virus. (d) Viral pellets are resuspended in appropriate media in 2000-fold less volume than the original supernatant.

### Example 2: In Vitro Enrichment of Jurkat Cells Using Dual Promoter Lentiviral Vector and MPA

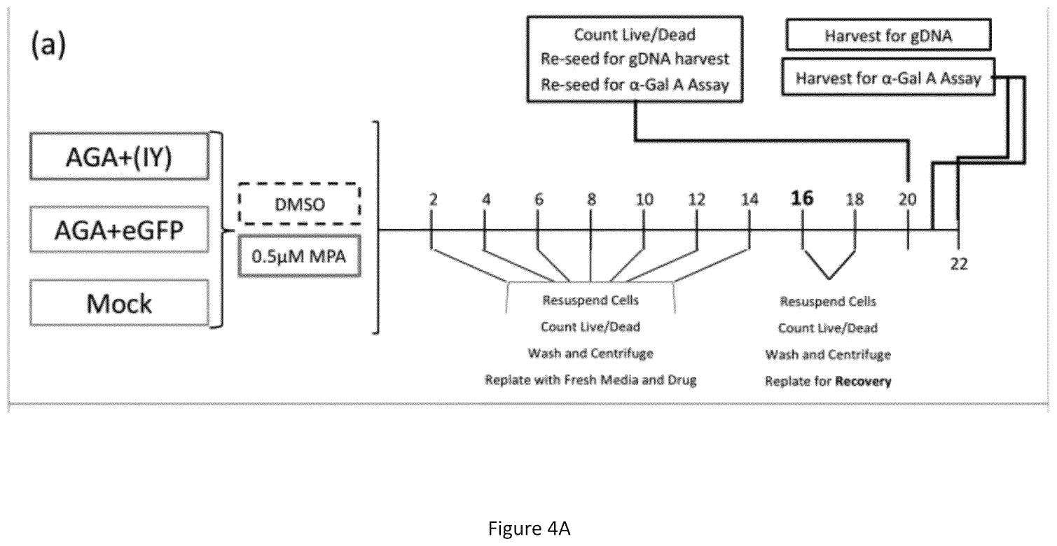

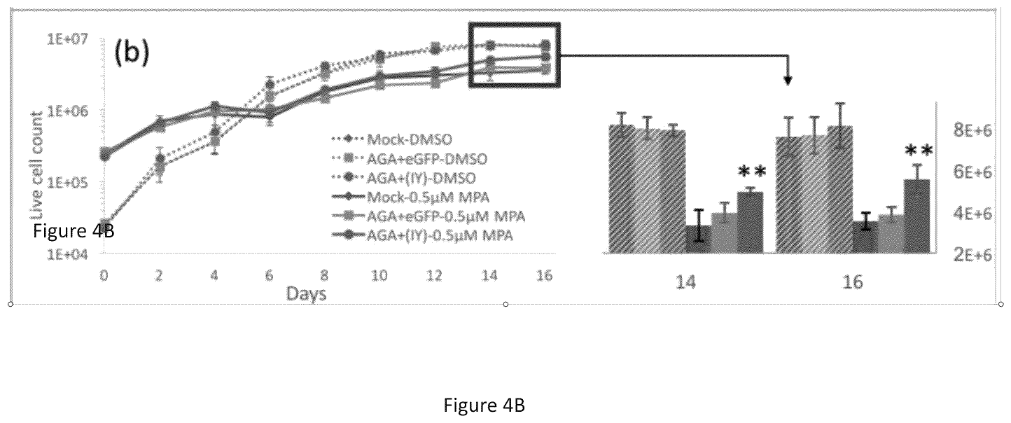

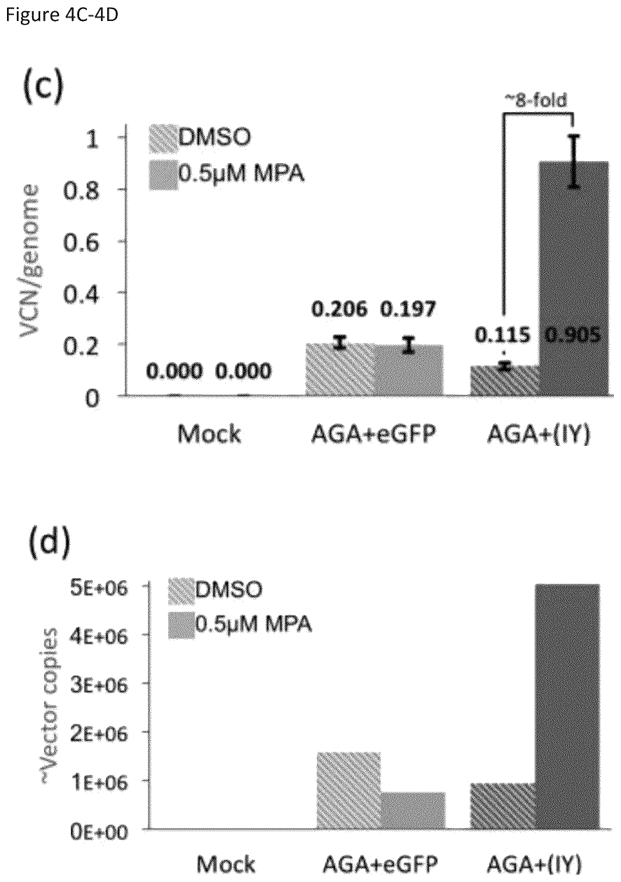

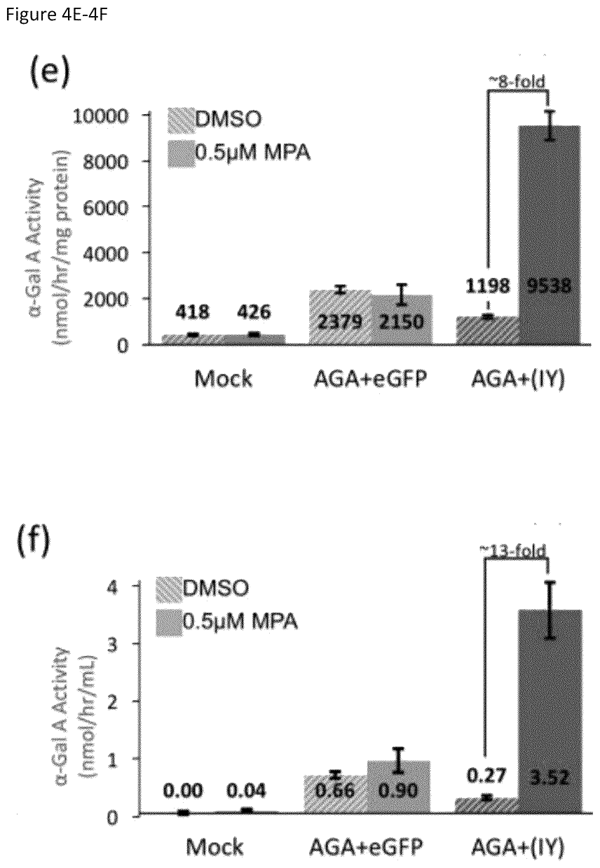

This Example demonstrates that cells expressing the dual promoter lentiviral vector of the present invention can be enriched by treatment of the cells with low doses of MPA in vitro. In this Example, Jurkat cells (immortalized T cells) are used. FIG. 4A shows a schematic for the experiment. 3 cell lines were generated by transducing Jurkat cells with the vectors described or with protamine sulphate only as a mock. VCN/genome was adjusted by dilution of the transduced cells with mock transduced cells. Each of these were seeded in 4 replicates and either treated with DMSO (vehicle) or 0.5 μM MPA. Fresh media with drug or vehicle was added after centrifuging the cells and discarding old media every two days. Samples were taken for cell counts. After 16 days of treatment, cells were reseeded for recovery in fresh media for a total of four days, after which they were counted and reseeded again for two days for α-gal A activity assay or one day for gDNA.

FIG. 4B shows cell counts for all groups. Dead cells were excluded using Trypan blue staining. The panel on the right shows an expanded view of days 14 and 16 (p<0.01, student's T Test compared to either negative control).

FIG. 4C shows results of vector copy number (VCN)/genome. VCN was determined by comparison to a standard curve using a qPCR based method and normalized to the number of beta actin (ACTB) copies to determine a per genome value. Patterned bars in the chart refer to samples treated with DMSO and solid bars refer to those treated with MPA. FIG. 4C shows about an 8-fold increase in the vector copy number of the total population per genome.

FIG. 4D demonstrates live vector copies after treatment. These were calculated by multiplying VCN/genome to live cell counts on Day 16. These values may be considered an approximation of vector positive cells after enrichment, assuming a majority of the transduced cells have one vector copy.

FIG. 4E and 4F demonstrate α-gal A activity. α-gal A activity was determined using 4-MUG substrate and normalized to protein as determined by BCA assay for cell lysates (4E) or to volume for culture supernatants (4F). As shown, there was a corresponding increase in α-gal A activity that corresponded with the increase in vector copy number per genome.

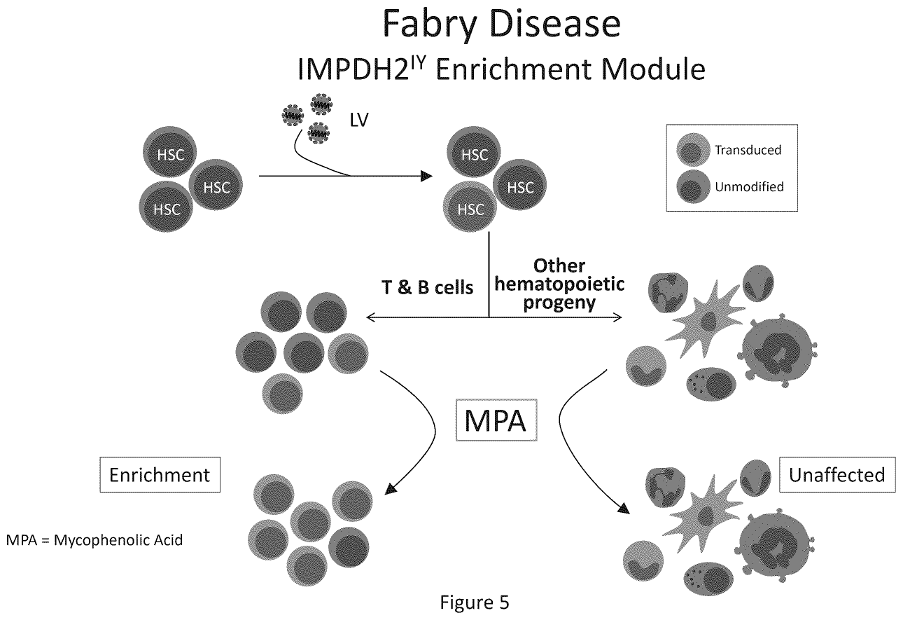

Thus, IMPDH2(IY) vector can be used to enrich for cells that express the transgene both in vitro and in vivo. FIG. 5 shows a schematic demonstrating how the IMPDH2(IY) enrichment would work using HSC cells.

### Example 3: Cell Enrichment In Vivo

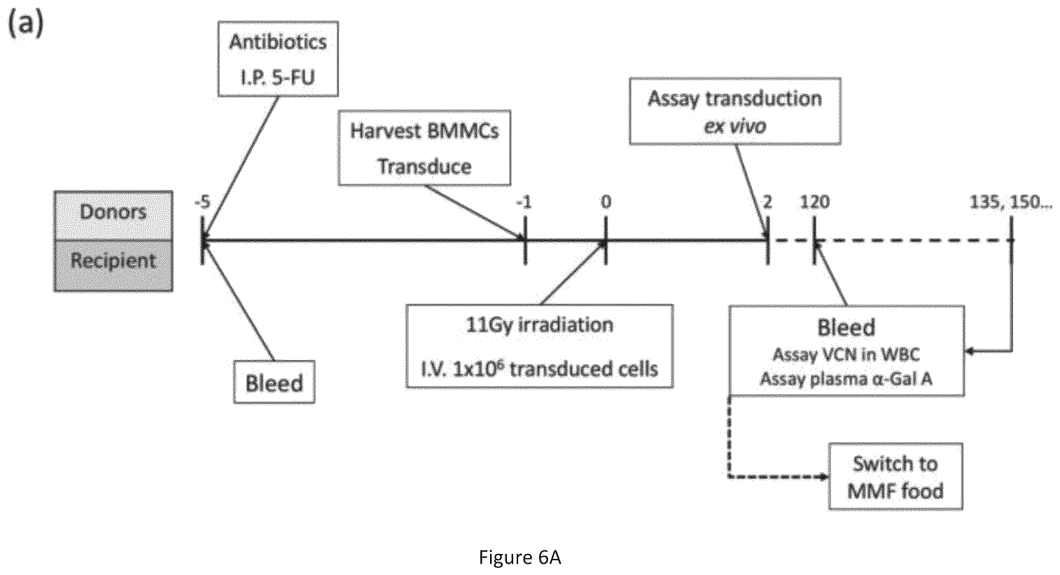

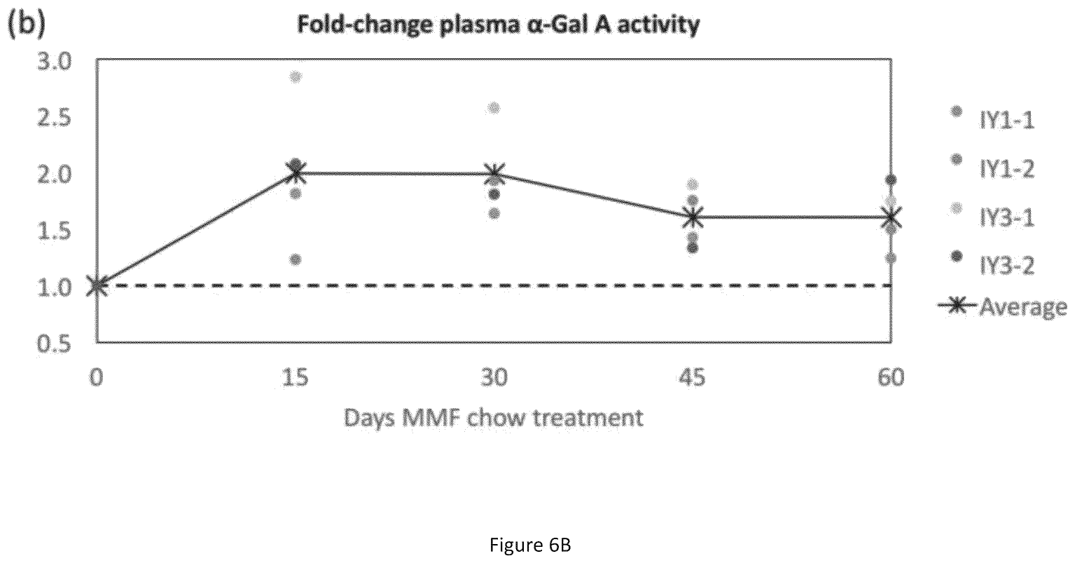

FIG. 6 shows the experimental schematic for testing enrichment as shown in FIG. 5 in vivo in a mouse model. An experimental schematic for hematopoietic stem progenitor cell (HSPC) engraftment is shown in FIG. 6A. Briefly, male and female Fabry mice aged 2-5 months of age are treated with 5-fluorouracil (5-FU) to deplete bone marrow cells and enrich for HSPCs. Four days after treatment these mice are euthanized, and bone marrow is isolated from femurs and tibia, and processed for transduction. Cells are cultured in stem cell media and transduced using lentivirus described above for 12 hours at a multiplicity of infection (MOI) of 10. Transduced cells are then harvested and prepared for administration to recipient mice. Recipients are male Fabry mice aged approximately 2 months. These are irradiated prior to engrafting transduced cells to deplete their own hematopoietic system. Some cells are retained in culture for an extra 48 hours to assay transduction. Mice are kept on prophylactic antibiotics as support during immune recovery. Engraftment of transduced cells is assessed by measuring VCN/genome in circulation using a qPCR-based assay and comparing to a standard curve, and by measuring plasma α-gal A activity. MMF medicated chow may be started after stable engraftment has been achieved, typically after 4 months. FIG. 6B shows four mice engrafted with LV/AGA+(IY) transduced cells. Engrafted mice were monitored for ˜9 months after engraftment to determine any gross negative effects of IMPDH2(IY) overexpression in the hematopoietic system. No obvious toxicities were observed. A baseline bleed was done to confirm engraftment and the mice were given 0.15% MMF capsule (TEVA generics, Israel) medicated chow ad libitum, and bled every 15 days to observe changes in plasma α-gal A activity, which is a measure of enrichment. Activity was normalized to baseline and are represented as fold-change. No drug-related toxicities were observed in 2 months of treatment.

### Example 4: Ex Vivo Gene Therapy for Fabry Disease

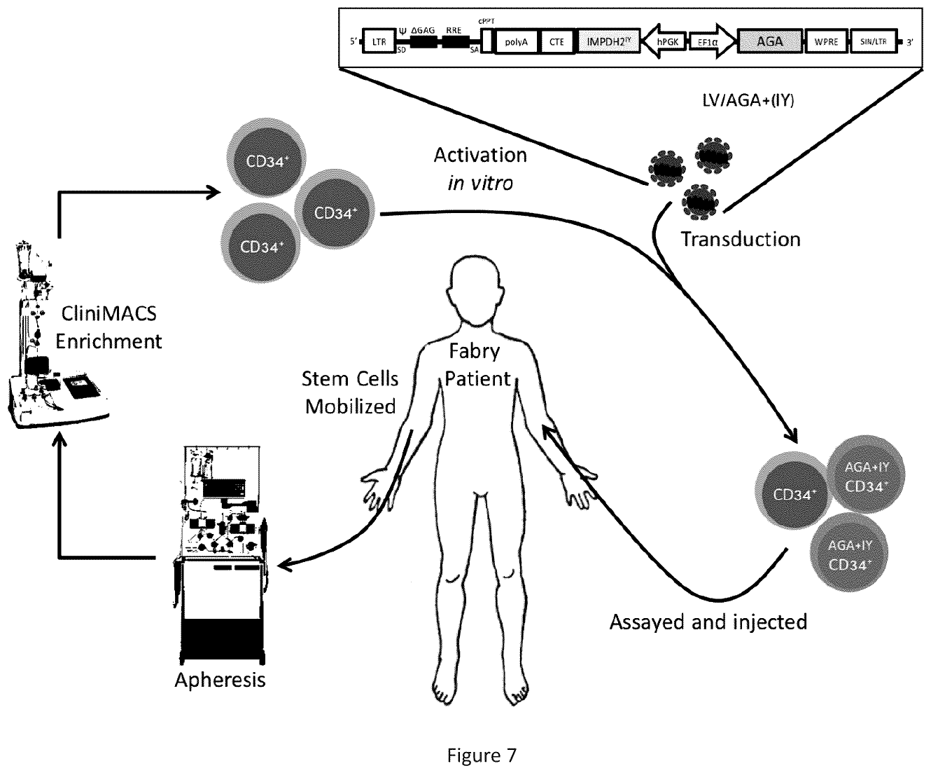

FIG. 7 demonstrates the overall protocol for treatment of a disorder by the methods described herein, specifically shows the steps for treatment of a lysosomal storage disorder Fabry disease.

Patient hematopoietic stem cells (HSCs) are mobilized to the peripheral blood by appropriate agents, for example G-CSF or plerixafor by known methods in the art. The patient is then subjected to apheresis to collect cells. Cells are then enriched for CD34, a cell surface marker of hematopoietic progenitor cells, using clinical magnet-assisted cell sorting (CliniMACS). The CD34+ cells are then cultured in the presence of cytokines and transduced using lentivirus ex vivo at an MOI of 1-10 for 12 hours, after which they are processed for cryopreservation. The patient may be conditioned using a low-dose regimen (30-50% of normal dose) of melphalan or other chemotherapeutic drug, after which they receive 2-10×10⁶ cells/kg intravenously. Patients are then monitored for hematopoietic reconstitution from transduced cells using plasma enzyme activity and vector copy number (VCN) per genome. Cell administration may be repeated after recovery from conditioning, as recommended by an appropriate physician.

Enrichment can be initiated if required by treatment with mycophenolate mofetil (MMF; CellCept, Roche, or approved generic); transduced T and B cells are resistant to the effects of this drug, providing them with a growth advantage. A low dose of oral MMF may be effective (0.1-5 mg/kg TID) but higher doses (5-10 mg/kg TID or 1000 mg BID) may also be tolerated, depending on the patient. As a general guideline, a blood concentration of 0.4-2 μM free mycophenolic acid (MPA) is desirable. MMF may be administered for the duration for which increased enzyme activity is desired, and doses adjusted to titrate the activity. MMF may also be substituted for MPA formulations (Myfortic, Novartis, or approved generic).

### Example 5: In Vitro Expression of GBA Protein in Transduced Cells

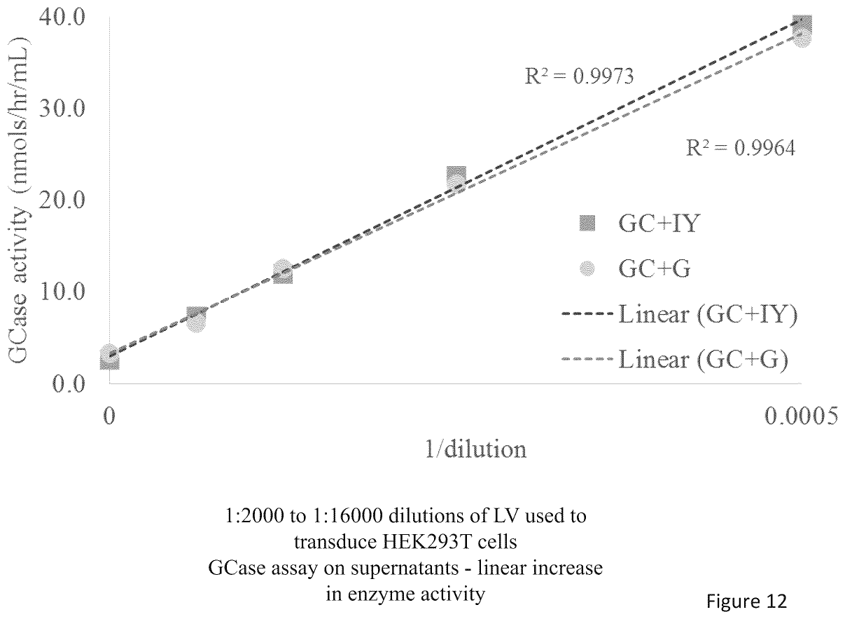

As demonstrated in FIGS. 12, 1:2000 to 1:16000 dilutions of dual promoter lentivirus vector also described in Example 6 that is able to express GBA protein was used to transduce HEK293T cells. Cell supernatant were then monitored for enzyme activity. GCase (GBA protein) assay was performed on supernatants, demonstrating a linear increase in enzyme activity.

### Example 6: Gaucher Disease

This Example demonstrates in vitro viral transduction followed by enrichment of Jurkat cells using the viral vectors for expression of GBA protein/enzyme, and increase in GBA enzyme activity in transduced cells.

A codon-optimized copy of the cDNA for GBA transgene was synthesized by GenScript. LV/GBA+(IY) was constructed by inserting the synthesized GBA transgene (SEQ ID NO: 6) into the dual promoter backbone (SEQ ID NO:2) at the EF1a locus using standard recombinant technology methods. A control vector with eGFP in place of IMPDH2(IY) was constructed using similar methods. Lentivirus was packaged as previously described in Example 1; briefly HEK293T were transiently transfected with packaging plasmids and the appropriate transfer vectors, and media replaced 16 hrs later. Supernatant containing viral particles were collected 40 and 64 hrs after transfection, filtered through a 0.22 μm membrane and concentrated by centrifugation. Pellets were resuspended in complete culture medium for Jurkat cells (RPMI-1640+10% FBS+Pen/strep/L-glutamine).

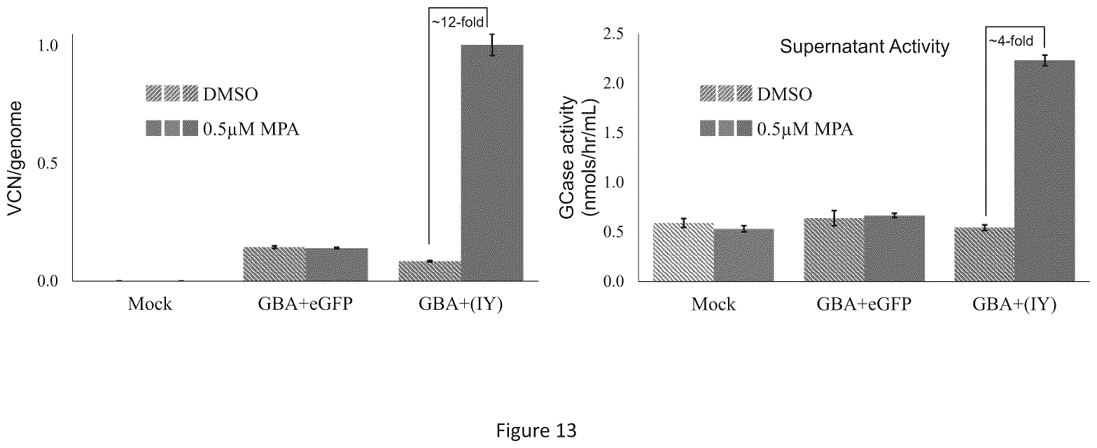

Jurkat cells were transduced with lentivirus at an MOI of ˜0.1-0.2. Cells were cultured for at least 2 weeks prior to measuring baseline vector copy number. Cell lines for each of LV/GBA+eGFP and LV/GBA+(IY), or non-transduced cells were produced in this way. To measure enrichment, testing was carried out as described in Example 2. Briefly, cells at known densities were seeded in quadruplicate for each cell line, and maintained in culture for 16 days with either vehicle (DMSO) or 0.5 μM MPA, with media being replaced every two days. After treatment, cells were diluted and recovered for 4-8 days. gDNA was extracted from cells sampled from each well, and vector copy number analyses were carried out using qPCR. Results are shown in FIG. 13. Cells from each well were also seeded at known and equal densities for 72 hours, after which supernatant was collected, cleared of cells and debris, and glucocerebrosidase activity measured using a fluorometric assay. Briefly, supernatants were incubated for 1 hour with substrate solution containing 4-methylumbelliferyl-beta-D-glucopyranoside (15 mM). The reaction was quenched with 0.1M glycine at pH 10 and fluorescence of the reaction product quantified against a calibration curve of the product. Results are shown in FIG. 13.

Each publication, patent, and patent publication cited in this disclosure is incorporated in reference herein in its entirety. The present invention is not intended to be limited to the foregoing examples but encompasses all such modifications and variations as come within the scope of the appended claims.

### Sequence Listing

The following sequences correspond with the plasmid maps in FIGS. 2A-2C.

### Genetic Elements of Plasmids and Lentivirus Vectors

f1 ori: Origin of plasmid replication in bacteria
Amp (R): Ampicillin-resistance gene (beta-lactamase)
5′ LTR: HIV1-derived 5′ Long Terminal Repeat: Viral element required for integration into host genome
3′ LTR/SIN: HIV1-derived 3′ Long Terminal Repeat with a 133 bp deletion to inactivate the capacity for any viral replication after retrotranscription in the host cell
Psi sequence: Retroviral Psi packaging element
5′Gag (del3rdG): Viral element required in 2^{nd} generation lentivirus systems for proviral RNA transcription
RRE: Viral REV response element, required for nuclear export of proviral RNA
cPPT: central polypurine tract, a lentiviral element that enhances nuclear import/export of viral RNA, consequently enhancing viral titer and transduction.
EF1a: Ubiquitous, constitutively expressing promoter derived from the human elongation factor 1 alpha gene
WPRE: Woodchuck Hepatitis post-transcriptional regulatory element; potently terminates transcription and stabilizes mRNA.
hPGK: Ubiquitous, constitutively expressing promoter derived from the human phosphoglycerate kinase 1 gene
CTE and polyA: C-Terminal end and polyA signal sequence for termination of transcription
coIMPDH2(IY): codon optimized transgene expressing the T333I, S351Y mutant of human 5′-inosine monophosphate dehydrogenase 2
AGA: Codon optimized transgene encoding human α-galactosidase A
GBA-codon optimized transgene for glucocerebrosidase (GBA protein)

>pDY DP Empty (SEQ ID NO: 1)
AAATTGTAAGCGTTAATATTTTGTTAAAATTCGCGTTAAATTTTTGTTAAATCAGC
TCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAA
GAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCA
CTAAATCGGAACCCTAAAGGGAGCCCCCGATTTAGAGCTTGACGGGGAAAGCCG
GCGAACGTGGCGAGAAAGGAAGGGAAGAAAGCGAAAGGAGCGGGCGCTAGGGC
GCTGGCAAGTGTAGCGGTCACGCTGCGCGTAACCACCACACCCGCCGCGCTTAA
TGCGCCGCTACAGGGCGCGTCCATTCGCCATTCAGGCTGCGCAACTGTTGGGAAG
GGCGATCGGTGCGGGCCTCTTCGCTATTACGCCAGCTGGCGAAAGGGGGATGTG
CTGCAAGGCGATTAAGTTGGGTAACGCCAGGGTTTTCCCAGTCACGACGTTGTAA
AACGACGGCCAGTGAATTGTAATACGACTCACTATAGGGCGAATTGGGCCCGAC
GTCGCATGCTTGGAAGGGCTAATTCACTCCCAAAGAAGACAAGATATCCTTGATC
TGTGGATCTACCACACACAAGGCTACTTCCCTGATTAGCAGAACTACACACCAGG
GCCAGGGGTCAGATATCCACTGACCTTTGGATGGTGCTACAAGCTAGTACCAGTT
GAGCCAGATAAGGTAGAAGAGGCCAATAAAGGAGAGAACACCAGCTTGTTACA
CCCTGTGAGCCTGCATGGGATGGATGACCCGGAGAGAGAAGTGTTAGAGTGGAG
GTTTGACAGCCGCCTAGCATTTCATCACGTGGCCCGAGAGCTGCATCCGGAGTAC
TTCAAGAACTGCTGATATCGAGCTTGCTACAAGGGACTTTCCGCTGGGGACTTTC
CAGGGAGGCGTGGCCTGGGCGGGACTGGGGAGTGGCGAGCCCTCAGATCCTGCA
TATAAGCAGCTGCTTTTTGCCTGTACTGGGTCTCTCTGGTTAGACCAGATCTGAGC
CTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTGGCGCCCGAAC
AGGGACTTGAAAGCGAAAGGGAAACCAGAGGAGCTCTCTCGACGCAGGACTCG
GCTTGCTGAAGCGCGCACGGCAAGAGGCGAGGGGCGGCGACTGGTGAGTACGCC
AAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAG
GGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACC
ACCGCACAGCAAGCGGCCGCTGATCTTCAGACCTGGAGGAGGAGATATGAGGGA
CAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAG
TGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGA
TACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGAT
TTGGAATCACACGACCTGGATGGAGTGGGACAGAGAAATTAACAATTACACAAG
CTTAATACACTCCTTAATTGAAGAATCGCAAAACCAGCAAGAAAAGAATGAACA
AGAATTATTGGAATTAGATAAATGGGCAAGTTTGTGGAATTGGTTTAACATAACA
AATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAG
TGAACGGATCTCGACGGGATCGATTTTAAAAGAAAAGGGGGGATTGGGGGGTAC
AGTGCAGGGGAAAGAATAGTAGACATAATAGCAACAGACATACAAACTAAAGA
ATTACAAAAACAAATTACAAAAATTCAAAATTTTATCGATAAGCTTTGCAAAGAT
GGATAAAGTTTTAAACAGAGAGGAATCTTTGCAGCTAATGGACCTTCTAGGTCTG
ACCCCGTACGCCTCGAGAGATCTGATCATAATCAGCCATACCACATTTGTAGAGG
TTTTACTTGCTTTAAAAAACCTCCCACACCTCCCCCTGAACCTGAAACATAAAAT
GAATGCAATTGTTGTTGTTAACTTGTTTATTGCAGCTTATAATGGTTACAAATAAG
GCAATAGCATCACAAATTTCACAAATAAGGCATTTTTTTCACTGCATTCTAGTTTT
GGTTTGTCCAAACTCATCAATGTATCTTATCATGTCTGGATCTCAAATCCCTCGGA
AGCTGCGCCTGTCTTAGGTTGGAGTGATACATTTTTATCACTTTTACCCGTCTTTG
GATTAGGCAGTAGCTCTGACGGCCCTCCTGTCTTAGGTTAGTGAAAAATGTCACT
CTCTTACCCGTCATTGGCTGTCCAGCTTAGCTCGCAGGGGAGGTGGTCTGCCTGC
AGGCGGATGGCGTTAACATATGACAACTTTCTCCCGGGTAATCTGACCGTTCGCT
AGCCCTGGGGAGAGAGGTCGGTGATTCGGTCAACGAGGGAGCCGACTGCCGACG
TGCGCTCCGGAGGCTTGCAGAATGCGGAACACCGCGCGGGCAGGAACAGGGCCC
ACACTACCGCCCCACACCCCGCCTCCCGCACCGCCCCTTCCCGGCCGCTGCTCTC
GGCGCGCCCCGCTGAGCAGCCGCTATTGGCCACAGCCCATCGCGGTCGGCGCGC
TGCCATTGCTCCCTGGCGCTGTCCGTCTGCGAGGGTACTAGTGAGACGTGCGGCT
TCCGTTTGTCACGTCCGGCACGCCGCGAACCGCAAGGAACCTTCCCGACTTAGGG
GCGGAGCAGGAAGCGTCGCCGGGGGGCCCACAAGGGTAGCGGCGAAGATCCGG
GTGACGCTGCGAACGGACGTGAAGAATGTGCGAGACCCAGGGTCGGCGCCGCTG
CGTTTCCCGGAACCACGCCCAGAGCAGCCGCGTCCCTGCGCAAACCCAGGGCTG
CCAAGGAAAAGGCGCAACCCCAACCCCGTGGTTAATTAAGGTGAAAGGAGTGGG
AATTGGCTCCGGTGCCCGTCAGTGGGCAGAGCGCACATCGCCCACAGTCCCCGA
GAAGTTGGGGGGAGGGGTCGGCAATTGAACCGGTGCCTAGAGAAGGTGGCGCG
GGGTAAACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCAACGGG
TTTGCCGCCAGAACACAGGTAAGTGCCGTGTGTGGTTCCCGCGGGCCTGGCCTCT
TTACGGGTTATGGCCCTTGCGTGCCTTGAATTACTTCCACCTGGCTGCAGTACGTG
ATTCTTGATCCCGAGCTTCGGGTTGGAAGTGGGTGGGAGAGTTCGAGGCCTTGCG
CTTAAGGAGCCCCTTCGCCTCGTGCTTGAGTTGAGGCCTGGCCTGGGCGCTGGGG
CCGCCGCGTGCGAATCTGGTGGCACCTTCGCGCCTGTCTCGCTGCTTTCGATAAG
TCTCTAGCCATTTAAAATTTTTGATGACCTGCTGCGACGCTTTTTTTCTGGCAAGA
TAGTCTTGTAAATGCGGGCCAAGATCTGCACACTGGTATTTCGGTTTTTGGGGCC
GCGGGCGGCGACGGGGCCCGTGCGTCCCAGCGCACATGTTCGGCGAGGCGGGGC
CTGCGAGCGCGGCCACCGAGAATCGGACGGGGGTAGTCTCAAGCTGGCCGGCCT
GCTCTGGTGCCTGGCCTCGCGCCGCCGTGTATCGCCCCGCCCTGGGCGGCAAGGC
TGGCCCGGTCGGCACCAGTTGCGTGAGCGGAAAGATGGCCGCTTCCCGGCCCTG
CTGCAGGGAGCTCAAAATGGAGGACGCGGCGCTCGGGAGAGCGGGCGGGTGAG
TCACCCACACAAAGGAAAAGGGCCTTTCCGTCCTCAGCCGTCGCTTCATGTGACT
CCACGGAGTACCGGGCGCCGTCCAGGCACCTCGATTAGTTCTCGAGCTTTTGGAG
TACGTCGTCTTTAGGTTGGGGGGAGGGGTTTTATGCGATGGAGTTTCCCCACACT
GAGTGGGTGGAGACTGAAGTTAGGCCAGCTTGGCACTTGATGTAATTCTCCTTGG
AATTTGCCCTTTTTGAGTTTGGATCTTGGTTCATTCTCAAGCCTCAGACAGTGGTT
CAAAGTTTTTTTCTTCCATTTCAGGTGTCGTGAGGAATTCTGCAGTCGACGGTACC
GCGGGCGCGCCCCGGGATCCAAGCTTCAATTGTGGTCACTCGACAATCAACCTCT
GGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAGGAGTTG
TGGCCCGTTGTCAGGCAACGTGGCGTGGTGTGCACTGTGTTTGCTGACGCAACCC
CCACTGGTTGGGGCATTGCCACCACCTGTCAGCTCCTTTCCGGGACTTTCGCTTTC
CCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAGCTGAC
GTCCTTTCCATGGCTGCTCGCCTGTGTTGCCACCTGGATTCTGCGCGGGACGTCCT
TCTGCTACGTCCCTTCGGCCCTCAATCCAGCGGACCTTCCTTCCCGCGGCCTGCTG
CCGGCTCTGCGGCCTCTTCCGCGTCTTCGCCTTCGCCCTCAGACGAGTCGGATCTC
CCTTTGGGCCGCCTCCCCGCCTGCTCGAGACCTAGAAAAACATGGAGCAATCAC
AAGTAGCAATACAGCAGCTACCAATGCTGATTGTGCCTGGCTAGAAGCACAAGA
GGAGGAGGAGGTGGGTTTTCCAGTCACACCTCAGGTACCTTTAAGACCAATGACT
TACAAGGCAGCTGTAGATCTTAGCCACTTTTTAAAAGAAAAGGGGGGACTGGAA
GGGCTAATTCACTCCCAACGAAGACAAGATCTGCTTTTTGCTTGTACTGGGTCTC
TCTGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAA
TCTCTAGCAGTAGTAGTTCATGTCATCTTATTATTCAGTATTTATAACTTGCAAAG
AAATGAATATCAGAGAGTGAGAGGACGCGTTGGATGCATAGCTTGAGTATTCTA
TAGTGTCACCTAAATAGCTTGGCGTAATCATGGTCATAGCTGTTTCCTGTGTGAA
ATTGTTATCCGCTCACAATTCCACACAACATACGAGCCGGAAGCATAAAGTGTAA
AGCCTGGGGTGCCTAATGAGTGAGCTAACTCACATTAATTGCGTTGCGCTCACTG
CCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCA
AAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCC
CTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTT
GGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAAT
AAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGT
CGTTTGGTATGGCTTCATTCAGCTCCGGTTCCCAACGATCAAGGCGAGTTACATG
ATCCCCCATGTTGTGCAAAAAAGCGGTTAGCTCCTTCGGTCCTCCGATCGTTGTC
AGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCG
TTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAAT
AAACAAATAGGGGTTCCGCGCACATTTCCCCGAAAAGTGCCACCTGATGCGGTG
TGAAATACCGCACAGATGCGTAAGGAGAAAATACCGCATCAGG
>pDY (hPGK-IMPDH2(IY)) EF1a EV (SEQ ID NO: 2)
AAATTGTAAGCGTTAATATTTTGTTAAAATTCGCGTTAAATTTTTGTTAAATCAGC
TCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAA
GAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCA
CTAAATCGGAACCCTAAAGGGAGCCCCCGATTTAGAGCTTGACGGGGAAAGCCG
GCGAACGTGGCGAGAAAGGAAGGGAAGAAAGCGAAAGGAGCGGGCGCTAGGGC
GCTGGCAAGTGTAGCGGTCACGCTGCGCGTAACCACCACACCCGCCGCGCTTAA
TGCGCCGCTACAGGGCGCGTCCATTCGCCATTCAGGCTGCGCAACTGTTGGGAAG
GGCGATCGGTGCGGGCCTCTTCGCTATTACGCCAGCTGGCGAAAGGGGGATGTG
CTGCAAGGCGATTAAGTTGGGTAACGCCAGGGTTTTCCCAGTCACGACGTTGTAA
AACGACGGCCAGTGAATTGTAATACGACTCACTATAGGGCGAATTGGGCCCGAC
GTCGCATGCTTGGAAGGGCTAATTCACTCCCAAAGAAGACAAGATATCCTTGATC
TGTGGATCTACCACACACAAGGCTACTTCCCTGATTAGCAGAACTACACACCAGG
GCCAGGGGTCAGATATCCACTGACCTTTGGATGGTGCTACAAGCTAGTACCAGTT
GAGCCAGATAAGGTAGAAGAGGCCAATAAAGGAGAGAACACCAGCTTGTTACA
CCCTGTGAGCCTGCATGGGATGGATGACCCGGAGAGAGAAGTGTTAGAGTGGAG
GTTTGACAGCCGCCTAGCATTTCATCACGTGGCCCGAGAGCTGCATCCGGAGTAC
TTCAAGAACTGCTGATATCGAGCTTGCTACAAGGGACTTTCCGCTGGGGACTTTC
CAGGGAGGCGTGGCCTGGGCGGGACTGGGGAGTGGCGAGCCCTCAGATCCTGCA
TATAAGCAGCTGCTTTTTGCCTGTACTGGGTCTCTCTGGTTAGACCAGATCTGAGC
CTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTGGCGCCCGAAC
AGGGACTTGAAAGCGAAAGGGAAACCAGAGGAGCTCTCTCGACGCAGGACTCG
GCTTGCTGAAGCGCGCACGGCAAGAGGCGAGGGGCGGCGACTGGTGAGTACGCC
AAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAG
GGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACC
ACCGCACAGCAAGCGGCCGCTGATCTTCAGACCTGGAGGAGGAGATATGAGGGA
CAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAG
TGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGA
TACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGAT
TTGGAATCACACGACCTGGATGGAGTGGGACAGAGAAATTAACAATTACACAAG
CTTAATACACTCCTTAATTGAAGAATCGCAAAACCAGCAAGAAAAGAATGAACA
AGAATTATTGGAATTAGATAAATGGGCAAGTTTGTGGAATTGGTTTAACATAACA
AATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAG
TGAACGGATCTCGACGGGATCGATTTTAAAAGAAAAGGGGGGATTGGGGGGTAC
AGTGCAGGGGAAAGAATAGTAGACATAATAGCAACAGACATACAAACTAAAGA
ATTACAAAAACAAATTACAAAAATTCAAAATTTTATCGATAAGCTTTGCAAAGAT
GGATAAAGTTTTAAACAGAGAGGAATCTTTGCAGCTAATGGACCTTCTAGGTCTG
ACCCCGTACGCCTCGAGAGATCTGATCATAATCAGCCATACCACATTTGTAGAGG
TTTTACTTGCTTTAAAAAACCTCCCACACCTCCCCCTGAACCTGAAACATAAAAT
GAATGCAATTGTTGTTGTTAACTTGTTTATTGCAGCTTATAATGGTTACAAATAAG
GCAATAGCATCACAAATTTCACAAATAAGGCATTTTTTTCACTGCATTCTAGTTTT
GGTTTGTCCAAACTCATCAATGTATCTTATCATGTCTGGATCTCAAATCCCTCGGA
AGCTGCGCCTGTCTTAGGTTGGAGTGATACATTTTTATCACTTTTACCCGTCTTTG
GATTAGGCAGTAGCTCTGACGGCCCTCCTGTCTTAGGTTAGTGAAAAATGTCACT
CTCTTACCCGTCATTGGCTGTCCAGCTTAGCTCGCAGGGGAGGTGGTCTGCCTGC
AGGTTAGAACAGTCTCTTTTCGTATGAGTGCAGTGAGTGGACGCCGCCTTCG
ACCTGGGCTGAAGAAGTTCTTTTCTCGAACTTCAGTTCGCCGGAATACATCA
TTGCCCGCACCTGTGTCAGGCTCTTAGCGCCGATATCCTGGCATGAATGCTG
AATTCCGGCGATCAGGTAAGGCACGAATTTGTGAATACTGCCCTTATCCTGG
ACAGCTCCAGACACGCCCTGTGCGACTTTGATCTTGTCTGCCTCGGAAAAAT
ACCTGTTCTGAGAGGACAGATGCTTATCCATGGCGTCCAGTGACCCCATGC
CCCTATATTTCTTCAGTCTGAACCCATCACTAAAGAAGTACTCGCCGGGGGC
TTCTGTGGTTGCAGCCAGCAGGCTGCCCATCATCACTGTGCTTGCCCCCAGA
GCCAGGGCTTTTGCGATGTGGCCCACATTCTGAATTCCCCCGTCAGCGATCA
CTGGGACTCCGAATCTCCGGGCATACTCGTACACCTTGTAGACAGCAGTTG
CCTGAGGTCGTCCACAGGCCAGCACTTCCTGAATGATGCAGATTGATCCACT
CCCCATTCCGACCCTCAGAGCATCCACTCCTGCGTCAATCAGGTTTTTGGCC
TGGGCTGCGGTCACGACATTGCCTCCGATGACCTGCAGATTTGGGTACTTG
TCCTTAATGTACTTGATCATATTAATCTGGAAGATGCTGTTTCCCTGGCTTG
AATCCAGCACGACCACGTCCACCCCTGCCTGAGCCAGCAGATCCAGGCGAT
ATTTATCGTCCTCGTGTGTGCCAATAGCGGCTCCACACAGCAGCTGTTTCTT
TGCGTCCTTACTAGCCAGAGGGTAATCTCGATTTTTCTTCAGGTCGGTGCGG
GCAATGATTGCCACCAGCTCATCGTCTTCATTCACGATAGGCAGTTTTCCTT
TCTTAGACCGCTGCAGAATCTCGTTGGCTTCCTTCAGTGTGATGCCGGCAGG
TGCGACCACCAGATCTTCGCGTTTGGTCATAATCTCTTCCAGAAAACAGTCA
TGCTCTTCCTCCTTCAGGAAATCGATGTCTCGACTAGAAATGATTCCCACCA
GTCGGCTGCCCATTCGTCCAGTATCTGTAATGGGGATGCCGCAAAATCCGT
GCCTAGCTTTGGCCTCGAACACATCGCGGACCCTGTCCTTGGGGCTCAGGA
CCACTGGGTCGGTGATAAAGCCCTGTTCGTATTTCTTCACCTTTCTGACCTC
ATTGGCCTGAAATTCTGGAGTGCAGTTATGGTGAATGAACCCGATCCCGCCT
GTCAGTGCCATAGCAATGGCCATGCCAGCCTCGGTGACAGTGTCCATAGGG
GAGCTCACCAGGGGTGTCTTCAGGGTGATTTTCTTGGTCAGGGCAGAAGTC
AGATCCACCTGGTCTGCGGTAAAATCAATATAGCCGGGCAGGATCAGGAAG
TCGTTGTAAGTCAGCCCGTCTCCACAATTAAACAGCTGCTGGGCGGTCAGTC
CATCATCAGGGACATAGGAAGTGCCTCCAGAAATCAGGTAGTCGGCCATGG
TGGCGCTAGCCCTGGGGAGAGAGGTCGGTGATTCGGTCAACGAGGGAGCCGACT
GCCGACGTGCGCTCCGGAGGCTTGCAGAATGCGGAACACCGCGCGGGCAGGAAC
AGGGCCCACACTACCGCCCCACACCCCGCCTCCCGCACCGCCCCTTCCCGGCCGC
TGCTCTCGGCGCGCCCCGCTGAGCAGCCGCTATTGGCCACAGCCCATCGCGGTCG
GCGCGCTGCCATTGCTCCCTGGCGCTGTCCGTCTGCGAGGGTACTAGTGAGACGT
GCGGCTTCCGTTTGTCACGTCCGGCACGCCGCGAACCGCAAGGAACCTTCCCGAC
TTAGGGGCGGAGCAGGAAGCGTCGCCGGGGGGCCCACAAGGGTAGCGGCGAAG
ATCCGGGTGACGCTGCGAACGGACGTGAAGAATGTGCGAGACCCAGGGTCGGCG
CCGCTGCGTTTCCCGGAACCACGCCCAGAGCAGCCGCGTCCCTGCGCAAACCCA
GGGCTGCCAAGGAAAAGGCGCAACCCCAACCCCGTGGTTAATTAAGGTGAAAGG
AGTGGGAATTGGCTCCGGTGCCCGTCAGTGGGCAGAGCGCACATCGCCCACAGT
CCCCGAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTCCCGAG
GGTGGGGGAGAACCGTATATAAGTGCAGTAGTCGCCGTGAACGTTCTTTTTCGCA
ACGGGTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGGCTGCAG
TACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCG
CTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGC
CGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGGCG
GGTGAGTCACCCACACAAAGGAAAAGGGCCTTTCCGTCCTCAGCCGTCGCTTCAT
GTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCC
CACACTGAGTGGGTGGAGACTGAAGTTAGGCCAGCTTGGCACTTGATGTAATTCT
CCTTGGAATTTGCCCTTTTTGAGTTTGGATCTTGGTTCATTCTCAAGCCTCAGACA
GTGGTTCAAAGTTTTTTTCTTCCATTTCAGGTGTCGTGAGGAATTCTGCAGTCGAC
GGTACCGCGGGCGCGCCCCGGGATCCAAGCTTCAATTGTGGTCACTCGACAATC
AACCTCTGGATTACAAAATTTGTGAAAGATTGACTGGTATTCTTAACTATGTTGC
TCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGA
CGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCG
CTGCTGGACAGGGGCTCGGCTGTTGGGCACTGACAATTCCGTGGTGTTGTCGGGG
AAGCTGACGTCCTTTCCATGGCTGCTCGCCTGTGTTGCCACCTGGATTCTGCGCG
GGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGA
GTCGGATCTCCCTTTGGGCCGCCTCCCCGCCTGCTCGAGACCTAGAAAAACATGG
AGCAATCACAAGTAGCAATACAGCAGCTACCAATGCTGATTGTGCCTGGCTAGA
AGCACAAGAGGAGGAGGAGGTGGGTTTTCCAGTCACACCTCAGGTACCTTTAAG
ACCAATGACTTACAAGGCAGCTGTAGATCTTAGCCACTTTTTAAAAGAAAAGGG
GGGACTGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTCAG
TGTGGAAAATCTCTAGCAGTAGTAGTTCATGTCATCTTATTATTCAGTATTTATAA
CTTGCAAAGAAATGAATATCAGAGAGTGAGAGGACGCGTTGGATGCATAGCTTG
AGTATTCTATAGTGTCACCTAAATAGCTTGGCGTAATCATGGTCATAGCTGTTTCC
TGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGC
GCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCG
CTCACTGACTCGCTGCGCTCGGTCGTTCGGCTGCGGCGAGCGGTATCAGCTCACT
CAAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCA
TAGTTGCCTGACTCCCCGTCGTGTAGATAACTACGATACGGGAGGGCTTACCATC
TGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGA
TCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGG
GAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTT
AGAAAAATAAACAAATAGGGGTTCCGCGCACATTTCCCCGAAAAGTGCCACCTG
ATGCGGTGTGAAATACCGCACAGATGCGTAAGGAGAAAATACCGCATCAGG
(BOLD: complementary sequence to IMPDH2(IY) transgene)(SEQ ID NO: 10)
>pDY AGA + (IY)(SEQ ID NO: 3)
AAATTGTAAGCGTTAATATTTTGTTAAAATTCGCGTTAAATTTTTGTTAAATCAGC
TCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAA
GAACGTGGACTCCAACGTCAAAGGGCGAAAAACCGTCTATCAGGGCGATGGCCC
ACTACGTGAACCATCACCCTAATCAAGTTTTTTGGGGTCGAGGTGCCGTAAAGCA
CTAAATCGGAACCCTAAAGGGAGCCCCCGATTTAGAGCTTGACGGGGAAAGCCG
GCGAACGTGGCGAGAAAGGAAGGGAAGAAAGCGAAAGGAGCGGGCGCTAGGGC
GCTGGCAAGTGTAGCGGTCACGCTGCGCGTAACCACCACACCCGCCGCGCTTAA
TGCGCCGCTACAGGGCGCGTCCATTCGCCATTCAGGCTGCGCAACTGTTGGGAAG
GGCGATCGGTGCGGGCCTCTTCGCTATTACGCCAGCTGGCGAAAGGGGGATGTG
CTGCAAGGCGATTAAGTTGGGTAACGCCAGGGTTTTCCCAGTCACGACGTTGTAA
AACGACGGCCAGTGAATTGTAATACGACTCACTATAGGGCGAATTGGGCCCGAC
GTCGCATGCTTGGAAGGGCTAATTCACTCCCAAAGAAGACAAGATATCCTTGATC
TGTGGATCTACCACACACAAGGCTACTTCCCTGATTAGCAGAACTACACACCAGG
GCCAGGGGTCAGATATCCACTGACCTTTGGATGGTGCTACAAGCTAGTACCAGTT
GAGCCAGATAAGGTAGAAGAGGCCAATAAAGGAGAGAACACCAGCTTGTTACA
CCCTGTGAGCCTGCATGGGATGGATGACCCGGAGAGAGAAGTGTTAGAGTGGAG
GTTTGACAGCCGCCTAGCATTTCATCACGTGGCCCGAGAGCTGCATCCGGAGTAC
TTCAAGAACTGCTGATATCGAGCTTGCTACAAGGGACTTTCCGCTGGGGACTTTC
CAGGGAGGCGTGGCCTGGGCGGGACTGGGGAGTGGCGAGCCCTCAGATCCTGCA
TATAAGCAGCTGCTTTTTGCCTGTACTGGGTCTCTCTGGTTAGACCAGATCTGAGC
CTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTGGCGCCCGAAC
AGGGACTTGAAAGCGAAAGGGAAACCAGAGGAGCTCTCTCGACGCAGGACTCG
GCTTGCTGAAGCGCGCACGGCAAGAGGCGAGGGGCGGCGACTGGTGAGTACGCC
AAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAG
GGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACC
ACCGCACAGCAAGCGGCCGCTGATCTTCAGACCTGGAGGAGGAGATATGAGGGA
CAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAG
TGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCAA
CTCACAGTCTGGGGCATCAAGCAGCTCCAGGCAAGAATCCTGGCTGTGGAAAGA
TACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGAT
TTGGAATCACACGACCTGGATGGAGTGGGACAGAGAAATTAACAATTACACAAG
CTTAATACACTCCTTAATTGAAGAATCGCAAAACCAGCAAGAAAAGAATGAACA
AGAATTATTGGAATTAGATAAATGGGCAAGTTTGTGGAATTGGTTTAACATAACA
AATTGGCTGTGGTATATAAAATTATTCATAATGATAGTAGGAGGCTTGGTAGGTT
TAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAG
TGAACGGATCTCGACGGGATCGATTTTAAAAGAAAAGGGGGGATTGGGGGGTAC
AGTGCAGGGGAAAGAATAGTAGACATAATAGCAACAGACATACAAACTAAAGA
ATTACAAAAACAAATTACAAAAATTCAAAATTTTATCGATAAGCTTTGCAAAGAT
GGATAAAGTTTTAAACAGAGAGGAATCTTTGCAGCTAATGGACCTTCTAGGTCTG
ACCCCGTACGCCTCGAGAGATCTGATCATAATCAGCCATACCACATTTGTAGAGG
TTTTACTTGCTTTAAAAAACCTCCCACACCTCCCCCTGAACCTGAAACATAAAAT
GAATGCAATTGTTGTTGTTAACTTGTTTATTGCAGCTTATAATGGTTACAAATAAG
GCAATAGCATCACAAATTTCACAAATAAGGCATTTTTTTCACTGCATTCTAGTTTT
GGTTTGTCCAAACTCATCAATGTATCTTATCATGTCTGGATCTCAAATCCCTCGGA
AGCTGCGCCTGTCTTAGGTTGGAGTGATACATTTTTATCACTTTTACCCGTCTTTG
GATTAGGCAGTAGCTCTGACGGCCCTCCTGTCTTAGGTTAGTGAAAAATGTCACT
CTCTTACCCGTCATTGGCTGTCCAGCTTAGCTCGCAGGGGAGGTGGTCTGCCTGC
AGGTTAGAACAGTCTCTTTTCGTATGAGTGCAGTGAGTGGACGCCGCCTTCG
ACCTGGGCTGAAGAAGTTCTTTTCTCGAACTTCAGTTCGCCGGAATACATCA
TTGCCCGCACCTGTGTCAGGCTCTTAGCGCCGATATCCTGGCATGAATGCTG
AATTCCGGCGATCAGGTAAGGCACGAATTTGTGAATACTGCCCTTATCCTGG
ACAGCTCCAGACACGCCCTGTGCGACTTTGATCTTGTCTGCCTCGGAAAAAT
ACCTGTTCTGAGAGGACAGATGCTTATCCATGGCGTCCAGTGACCCCATGC
CCCTATATTTCTTCAGTCTGAACCCATCACTAAAGAAGTACTCGCCGGGGGC
TTCTGTGGTTGCAGCCAGCAGGCTGCCCATCATCACTGTGCTTGCCCCCAGA
GCCAGGGCTTTTGCGATGTGGCCCACATTCTGAATTCCCCCGTCAGCGATCA
CTGGGACTCCGAATCTCCGGGCATACTCGTACACCTTGTAGACAGCAGTTG
CCTGAGGTCGTCCACAGGCCAGCACTTCCTGAATGATGCAGATTGATCCACT
CCCCATTCCGACCCTCAGAGCATCCACTCCTGCGTCAATCAGGTTTTTGGCC
TGGGCTGCGGTCACGACATTGCCTCCGATGACCTGCAGATTTGGGTACTTG
TCCTTAATGTACTTGATCATATTAATCTGGAAGATGCTGTTTCCCTGGCTTG
AATCCAGCACGACCACGTCCACCCCTGCCTGAGCCAGCAGATCCAGGCGAT
ATTTATCGTCCTCGTGTGTGCCAATAGCGGCTCCACACAGCAGCTGTTTCTT
TGCGTCCTTACTAGCCAGAGGGTAATCTCGATTTTTCTTCAGGTCGGTGCGG
GCAATGATTGCCACCAGCTCATCGTCTTCATTCACGATAGGCAGTTTTCCTT
TCTTAGACCGCTGCAGAATCTCGTTGGCTTCCTTCAGTGTGATGCCGGCAGG
TGCGACCACCAGATCTTCGCGTTTGGTCATAATCTCTTCCAGAAAACAGTCA
TGCTCTTCCTCCTTCAGGAAATCGATGTCTCGACTAGAAATGATTCCCACCA
GTCGGCTGCCCATTCGTCCAGTATCTGTAATGGGGATGCCGCAAAATCCGT
GCCTAGCTTTGGCCTCGAACACATCGCGGACCCTGTCCTTGGGGCTCAGGA
CCACTGGGTCGGTGATAAAGCCCTGTTCGTATTTCTTCACCTTTCTGACCTC
ATTGGCCTGAAATTCTGGAGTGCAGTTATGGTGAATGAACCCGATCCCGCCT
GTCAGTGCCATAGCAATGGCCATGCCAGCCTCGGTGACAGTGTCCATAGGG
GAGCTCACCAGGGGTGTCTTCAGGGTGATTTTCTTGGTCAGGGCAGAAGTC
AGATCCACCTGGTCTGCGGTAAAATCAATATAGCCGGGCAGGATCAGGAAG
TCGTTGTAAGTCAGCCCGTCTCCACAATTAAACAGCTGCTGGGCGGTCAGTC
CATCATCAGGGACATAGGAAGTGCCTCCAGAAATCAGGTAGTCGGCCATGG
TGGCGCTAGCCCTGGGGAGAGAGGTCGGTGATTCGGTCAACGAGGGAGCCGACT
GCCGACGTGCGCTCCGGAGGCTTGCAGAATGCGGAACACCGCGCGGGCAGGAAC
AGGGCCCACACTACCGCCCCACACCCCGCCTCCCGCACCGCCCCTTCCCGGCCGC
TGCTCTCGGCGCGCCCCGCTGAGCAGCCGCTATTGGCCACAGCCCATCGCGGTCG
GCGCGCTGCCATTGCTCCCTGGCGCTGTCCGTCTGCGAGGGTACTAGTGAGACGT
GCGGCTTCCGTTTGTCACGTCCGGCACGCCGCGAACCGCAAGGAACCTTCCCGAC
TTAGGGGCGGAGCAGGAAGCGTCGCCGGGGGGCCCACAAGGGTAGCGGCGAAG
ATCCGGGTGACGCTGCGAACGGACGTGAAGAATGTGCGAGACCCAGGGTCGGCG
CCGCTGCGTTTCCCGGAACCACGCCCAGAGCAGCCGCGTCCCTGCGCAAACCCA
GGGCTGCCAAGGAAAAGGCGCAACCCCAACCCCGTGGTTAATTAAGGTGAAAGG
AGTGGGAATTGGCTCCGGTGCCCGTCAGTGGGCAGAGCGCACATCGCCCACAGT
CCCCGAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTCCCGAG
GGTGGGGGAGAACCGTATATAAGTGCAGTAGTCGCCGTGAACGTTCTTTTTCGCA
ACGGGTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGGCTGCAG
TACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCCTGGGCG
CTGGGGCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGC
CGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGGCG
GGTGAGTCACCCACACAAAGGAAAAGGGCCTTTCCGTCCTCAGCCGTCGCTTCAT
GTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCC
CACACTGAGTGGGTGGAGACTGAAGTTAGGCCAGCTTGGCACTTGATGTAATTCT
CCTTGGAATTTGCCCTTTTTGAGTTTGGATCTTGGTTCATTCTCAAGCCTCAGACA
GTGGTTCAAAGTTTTTTTCTTCCATTTCAGGTGTCGTGAGGAATTCGCCACCATGC
AACTTCGAAACCCAGAGCTCCACCTCGGATGTGCCCTTGCTCTGAGGTTCCTGGCGCT
GGTGTCTTGGGATATACCCGGAGCACGCGCTCTGGACAACGGGCTGGCCCGGACTCC
AACCATGGGTTGGCTCCATTGGGAAAGGTTTATGTGCAACTTGGACTGCCAGGAAGAA
CCCGACTCCTGTATTTCCGAGAAACTCTTCATGGAGATGGCCGAGCTGATGGTTAGCG
AAGGCTGGAAGGATGCCGGTTATGAATACTTGTGTATCGACGATTGTTGGATGGCTCC
CCAGCGGGACAGTGAAGGACGACTCCAGGCAGATCCGCAACGGTTCCCTCATGGCAT
ACGGCAGCTCGCCAATTACGTGCACAGCAAGGGTTTGAAGCTGGGGATATATGCTGAC
GTGGGCAACAAAACCTGTGCTGGTTTCCCCGGCAGCTTCGGCTACTATGATATAGATG
CACAAACCTTCGCTGATTGGGGCGTGGACCTGCTTAAATTTGACGGCTGTTACTGCGA
CAGCTTGGAAAACCTCGCCGATGGATATAAACACATGAGCCTTGCACTCAATCGGACT
GGCCGGAGCATTGTCTACTCTTGCGAGTGGCCATTGTACATGTGGCCTTTCCAGAAGC
CTAACTATACGGAGATTAGACAGTATTGTAATCACTGGAGAAACTTTGCAGATATCGAC
GACTCATGGAAGTCCATCAAATCTATTCTGGACTGGACTTCATTCAATCAGGAGCGCAT
CGTCGATGTTGCCGGTCCAGGTGGATGGAACGACCCTGACATGCTCGTAATTGGGAAT
TTCGGACTGTCCTGGAATCAGCAGGTCACACAGATGGCTTTGTGGGCTATCATGGCAG
CCCCACTCTTTATGTCTAACGATTTGCGGCATATTTCACCACAGGCCAAAGCCCTGCTG
CAAGATAAGGACGTCATAGCGATTAACCAGGACCCACTGGGAAAGCAGGGCTACCAG
CTGAGACAGGGCGACAATTTTGAGGTCTGGGAAAGACCTCTTAGCGGGCTGGCGTGG
GCCGTAGCCATGATTAATCGCCAGGAAATTGGCGGCCCTCGCTCTTACACTATCGCGG
TCGCCAGTCTGGGCAAGGGAGTCGCTTGTAACCCCGCCTGCTTCATAACTCAGTTGCT
GCCCGTGAAACGGAAGCTGGGCTTCTATGAATGGACTAGCAGACTCCGCAGTCATATT
AATCCGACTGGTACGGTGCTGCTGCAACTGGAGAATACCATGCAGATGTCACTTAAGG
ATCTTCTGTGAGAACCCGGGATCCAAGCTTCAATTGTGGTCACTCGACAATCAACC
TCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGA
GTTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGC
TGGACAGGGGCTCGGCTGTTGGGCACTGACAATTCCGTGGTGTTGTCGGGGAAG
CTGACGTCCTTTCCATGGCTGCTCGCCTGTGTTGCCACCTGGATTCTGCGCGGGAC
GTCCTTCTGCTACGTCCCTTCGGCCCTCAATCCAGCGGACCTTCCTTCCCGCGGCC
TGCTGCCGGCTCTGCGGCCTCTTCCGCGTCTTCGCCTTCGCCCTCAGACGAGTCG
GATCTCCCTTTGGGCCGCCTCCCCGCCTGCTCGAGACCTAGAAAAACATGGAGCA
ATCACAAGTAGCAATACAGCAGCTACCAATGCTGATTGTGCCTGGCTAGAAGCA
CAAGAGGAGGAGGAGGTGGGTTTTCCAGTCACACCTCAGGTACCTTTAAGACCA
ATGACTTACAAGGCAGCTGTAGATCTTAGCCACTTTTTAAAAGAAAAGGGGGGA
CTGGAAGGGCTAATTCACTCCCAACGAAGACAAGATCTGCTTTTTGCTTGTACTG
GGTCTCTCTGGTTAGACCAGATCTGAGCCTGGGAGCTCTCTGGCTAACTAGGGAA
CCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTG
GAAAATCTCTAGCAGTAGTAGTTCATGTCATCTTATTATTCAGTATTTATAACTTG
CAAAGAAATGAATATCAGAGAGTGAGAGGACGCGTTGGATGCATAGCTTGAGTA
TTCTATAGTGTCACCTAAATAGCTTGGCGTAATCATGGTCATAGCTGTTTCCTGTG
TGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTC
ACTGCCCGCTTTCCAGTCGGGAAACCTGTCGTGCCAGCTGCATTAATGAATCGGC
CAACGCGCGGGGAGAGGCGGTTTGCGTATTGGGCGCTCTTCCGCTTCCTCGCTCA
CTGACTCGCTGCGCTCGGTCGTTCGGCTGCGGCGAGCGGTATCAGCTCACTCAAA
GGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAG
AGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTT
TCTCCCTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTG
GTCATGAGATTATCAAAAAGGATCTTCACCTAGATCCTTTTAAATTAAAAATGAA
GTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTCGCCAG
TTAATAGTTTGCGCAACGTTGTTGCCATTGCTACAGGCATCGTGGTGTCACGCTC
GTCGTTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGG
AAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAG
CATTTATCAGGGTTATTGTCTCATGAGCGGATACATATTTGAATGTATTTAGAAA
AATAAACAAATAGGGGTTCCGCGCACATTTCCCCGAAAAGTGCCACCTGATGCG
GTGTGAAATACCGCACAGATGCGTAAGGAGAAAATACCGCATCAGG
BOLD: complementary sequence to IMPDH2(IY)(SEQ ID NO: 10)
Italics: AGA transgene
Plasmid expressing GBA + IY (SEQ ID NO: 4)
CCGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAGGTAAGTGCCGTG
TGTGGTTCCCGCGGGCCTGGCCTCTTTACGGGTTATGGCCCTTGCGTGCCTTGAAT
TACTTCCACCTGGCTGCAGTACGTGATTCTTGATCCCGAGCTTCGGGTTGGAAGT
GGGTGGGAGAGTTCGAGGCCTTGCGCTTAAGGAGCCCCTTCGCCTCGTGCTTGAG
TTGAGGCCTGGCCTGGGCGCTGGGGCCGCCGCGTGCGAATCTGGTGGCACCTTCG
CGCCTGTCTCGCTGCTTTCGATAAGTCTCTAGCCATTTAAAATTTTTGATGACCTG
CTGCGACGCTTTTTTTCTGGCAAGATAGTCTTGTAAATGCGGGCCAAGATCTGCA
CACTGGTATTTCGGTTTTTGGGGCCGCGGGCGGCGACGGGGCCCGTGCGTCCCAG
CGCACATGTTCGGCGAGGCGGGGCCTGCGAGCGCGGCCACCGAGAATCGGACGG
GGGTAGTCTCAAGCTGGCCGGCCTGCTCTGGTGCCTGGCCTCGCGCCGCCGTGTA
TCGCCCCGCCCTGGGCGGCAAGGCTGGCCCGGTCGGCACCAGTTGCGTGAGCGG
AAAGATGGCCGCTTCCCGGCCCTGCTGCAGGGAGCTCAAAATGGAGGACGCGGC
GCTCGGGAGAGCGGGCGGGTGAGTCACCCACACAAAGGAAAAGGGCCTTTCCGT
CCTCAGCCGTCGCTTCATGTGACTCCACGGAGTACCGGGCGCCGTCCAGGCACCT
CGATTAGTTCTCGAGCTTTTGGAGTACGTCGTCTTTAGGTTGGGGGGAGGGGTTT
TATGCGATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCTTGGTTC
ATTCTCAAGCCTCAGACAGTGGTTCAAAGTTTTTTTCTTCCATTTCAGGTGTCGTG
AGGAATTCGCTAGCGCCACCATGGAGTTCTCAAGCCCCTCTCGGGAAGAATGCCCAA
AACCTCTGTCACGGGTGTCTATCATGGCTGGATCACTGACTGGCCTGCTGCTGCTGCA
GGCCGTGAGCTGGGCCTCCGGAGCCCGGCCTTGCATCCCAAAGTCTTTCGGCTACAG
CTCCGTGGTGTGCGTGTGCAACGCCACCTATTGTGACTCCTTCGATCCCCCTACCTTT
CCCGCCCTGGGCACATTTTCTCGGTACGAGTCTACACGCAGCGGCAGGAGAATGGAG
CTGAGCATGGGCCCTATCCAGGCCAATCACACCGGAACAGGCCTGCTGCTGACCCTG
CAGCCAGAGCAGAAGTTCCAGAAGGTGAAGGGCTTTGGAGGAGCAATGACAGACGCA
GCCGCCCTGAACATCCTGGCCCTGTCCCCACCCGCCCAGAATCTGCTGCTGAAGTCC
TACTTCTCTGAGGAGGGCATCGGCTATAACATCATCAGGGTGCCCATGGCCAGCTGCG
ACTTTTCCATCAGAACCTACACATATGCCGATACCCCTGACGATTTCCAGCTGCACAAT
TTTTCCCTGCCAGAGGAGGATACAAAGCTGAAGATCCCACTGATCCACAGGGCCCTGC
AGCTGGCCCAGAGGCCCGTGAGCCTGCTGGCCAGCCCCTGGACCTCCCCTACATGG
CTGAAGACCAACGGCGCCGTGAATGGCAAGGGCTCTCTGAAGGGACAGCCAGGCGA
CATCTACCACCAGACATGGGCCCGCTATTTCGTGAAGTTTCTGGATGCCTACGCCGAG
CACAAGCTGCAGTTCTGGGCCGTGACCGCAGAGAACGAGCCTTCTGCCGGCCTGCTG
AGCGGCTATCCCTTCCAGTGCCTGGGCTTTACACCTGAGCACCAGAGGGACTTTATCG
CCAGAGATCTGGGCCCAACCCTGGCCAACTCCACACACCACAATGTGCGGCTGCTGA
TGCTGGACGATCAGCGCCTGCTGCTGCCTCACTGGGCCAAGGTGGTGCTGACCGACC
CAGAGGCCGCCAAGTACGTGCACGGCATCGCCGTGCACTGGTATCTGGATTTCCTGG
CACCAGCAAAGGCCACCCTGGGAGAGACACACAGGCTGTTCCCTAACACCATGCTGTT
TGCCAGCGAGGCCTGCGTGGGCTCCAAGTTTTGGGAGCAGTCCGTGCGGCTGGGCT
CTTGGGACAGGGGCATGCAGTACTCCCACTCTATCATCACCAATCTGCTGTATCACGT
GGTGGGCTGGACAGACTGGAACCTGGCCCTGAATCCAGAGGGCGGCCCCAACTGGG
TGAGAAATTTCGTGGATAGCCCCATCATCGTGGACATCACCAAGGATACATTCTACAAG
CAGCCAATGTTTTATCACCTGGGCCACTTCTCTAAGTTTATCCCAGAGGGCAGCCAGA
GGGTGGGCCTGGTGGCCAGCCAGAAGAACGACCTGGATGCAGTGGCCCTGATGCAC
CCTGACGGCTCCGCCGTGGTGGTGGTGCTGAATCGCTCTAGCAAGGACGTGCCTCTG
ACCATCAAGGACCCCGCCGTGGGCTTTCTGGAGACCATTTCACCCGGCTATTCTATTC
ATACCTATCTGTGGAGGAGGCAGTAACCTGCAGGGGATCCAAGCTTCAATTGTGGT
CACTCGACAATCAACCTCTGGATTACAAAATTTGTGAAAGATTGACTGGTATTCT
TAACTATGTTGCTCCTTTTACGCTATGTGGATACGCTGCTTTAATGCCTTTGTATC
ATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCAC
TGTGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCCGC
CTGCCTTGCCCGCTGCTGGACAGGGGCTCGGCTGTTGGGCACTGACAATTCCGTG
GTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTC
GCCCTCAGACGAGTCGGATCTCCCTTTGGGCCGCCTCCCCGCCTGCTCGAGACCT
AGAAAAACATGGAGCAATCACAAGTAGCAATACAGCAGCTACCAATGCTGATTG
TGCCTGGCTAGAAGCACAAGAGGAGGAGGAGGTGGGTTTTCCAGTCACACCTCA
GGTACCTTTAAGACCAATGACTTACAAGGCAGCTGTAGATCTTAGCCACTTTTTA
AAAGAAAAGGGGGGACTGGAAGGGCTAATTCACTCCCAACGAAGACAAGATCT
GCTTTTTGCTTGTACTGGGTCTCTCTGGTTAGACCAGATCTGAGCCTGGGAGCTCT
CTGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAGA
CCCTTTTAGTCAGTGTGGAAAATCTCTAGCAGTAGTAGTTCATGTCATCTTATTAT
TCAGTATTTATAACTTGCAAAGAAATGAATATCAGAGAGTGAGAGGACGCGTTG
GATGCATAGCTTGAGTATTCTATAGTGTCACCTAAATAGCTTGGCGTAATCATGG
TCATAGCTGTTTCCTGTGTGAAATTGTTATCCGCTCACAATTCCACACAACATACG
AGCCGGAAGCATAAAGTGTAAAGCCTGGGGTGCCTAATGAGTGAGCTAACTCAC
ATTAATTGCGTTGCGCTCACTGCCCGCTTTCCAGTCGGGAAACCTGTCGTGCCAG
CTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAG
GATTAGCAGAGCGAGGTATGTAGGCGGTGCTACAGAGTTCTTGAAGTGGTGGCC
TAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATC
TCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCTA
TTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACC
GGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGT
GACTGGTGAGTACTCAACCAAGTCATTCTGAGAATAGTGTATGCGGCGACCGAG
TTGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTAC
CGCTGTTGAGATCCAGTTCGATGTAACCCACTCGTGCACCCAACTGATCTTCAGC
ATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTT
TTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAA
AGTGCCACCTGATGCGGTGTGAAATACCGCACAGATGCGTAAGGAGAAAATACC
GCATCAGGAAATTGTAAGCGTTAATATTTTGTTAAAATTCGCGTTAAATTTTTGTT
AAATCAGCTCATTTTTTAACCAATAGGCCGAAATCGGCAAAATCCCTTATAAATC
AAAAGAATAGACCGAGATAGGGTTGAGTGTTGTTCCAGTTTGGAACAAGAGTCC
ACTATTAAAGAACGTGGACTCCAACGTCAAAGGGCGAAAAACCGTCTATCAGGG
CGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGG
GGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCG
CCGCGCTTAATGCGCCGCTACAGGGCGCGTCCATTCGCCATTCAGGCTGCGCAAC
TGTTGGGAAGGGCGATCGGTGCGGGCCTCTTCGCTATTACGCCAGCTGGCGAAA
GGGGGATGTGCTGCAAGGCGATTAAGTTGGGTAACGCCAGGGTTTTCCCAGTCA
CGACGTTGTAAAACGACGGCCAGTGAATTGTAATACGACTCACTATAGGGCGAA
TTGGGCCCGACGTCGCATGCTTGGAAGGGCTAATTCACTCCCAAAGAAGACAAG
ATATCCTTGATCTGTGGATCTACCACACACAAGGCTACTTCCCTGATTAGCAGAA
CTACACACCAGGGCCAGGGGTCAGATATCCACTGACCTTTGGATGGTGCTACAA
GCTAGTACCAGTTGAGCCAGATAAGGTAGAAGAGGCCAATAAAGGAGAGAACA
CCAGCTTGTTACACCCTGTGAGCCTGCATGGGATGGATGACCCGGAGAGAGAAG
TGTTAGAGTGGAGGTTTGACAGCCGCCTAGCATTTCATCACGTGGCCCGAGAGCT
GCATCCGGAGTACTTCAAGAACTGCTGATATCGAGCTTGCTACAAGGGACTTTCC
GCTGGGGACTTTCCAGGGAGGCGTGGCCTGGGCGGGACTGGGGAGTGGCGAGCC
CTCAGATCCTGCATATAAGCAGCTGCTTTTTGCCTGTACTGGGTCTCTCTGGTTAG
ACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACT
CTGGTAACTAGAGATCCCTCAGACCCTTTTAGTCAGTGTGGAAAATCTCTAGCAG
TGGCGCCCGAACAGGGACTTGAAAGCGAAAGGGAAACCAGAGGAGCTCTCTCGA
CGCAGGACTCGGCTTGCTGAAGCGCGCACGGCAAGAGGCGAGGGGCGGCGACT
GGTGAGTACGCCAAAAATTTTGACTAGCGGAGGCTAGAAGGAGAGAGATGGGTG
CGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAA
GCAGGGAGCTAGAACGATTCGCAGTTAATCCTGGCCTGTTAGAAACATCAGAAG
GCTGTAGACAAATACTGGGACAGCTACAACCATCCCTTCAGACAGGATCAGAAG
AACTTAGATCATTATATAATACAGTAGCAACCCTCTATTGTGTGCATCAAAGGAT
AGAGATAAAAGACACCAAGGAAGCTTTAGACAAGATAGAGGAAGAGCAAAACA
AAAGTAAGACCACCGCACAGCAAGCGGCCGCTGATCTTCAGACCTGGAGGAGGA
GATATGAGGGACAATTGGAGAAGTGAATTATATAAATATAAAGTAGTAAAAATT
GAACCATTAGGAGTAGCACCCACCAAGGCAAAGAGAAGAGTGGTGCAGAGAGA
AAAAAGAGCAGTGGGAATAGGAGCTTTGTTCCTTGGGTTCTTGGGAGCAGCAGG
AAGCACTATGGGCGCAGCGTCAATGACGCTGACGGTACAGGCCAGACAATTATT
GTCTGGTATAGTGCAGCAGCAGAACAATTTGCTGAGGGCTATTGAGGCGCAACA
GCATCTGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTGCTCTG
GAAAACTCATTTGCACCACTGCTGTGCCTTGGAATGCTAGTTGGAGTAATAAATC
TCTGGAACAGATTTGGAATCACACGACCTGGATGGAGTGGGACAGAGAAATTAA
CAATTACACAAGCTTAATACACTCCTTAATTGAAGAATCGCAAAACCAGCAAGA
AAAGAATGAACAAGAATTATTGGAATTAGATAAATGGGCAAGTTTGTGGAATTG
GTTTAACATAACAAATTGGCTGTGGTATATAAAATTATTCATAATGATAGTAGGA
GGCTTGGTAGGTTTAAGAATAGTTTTTGCTGTACTTTCTATAGTGAATAGAGTTA
GGCAGGGATATTCACCATTATCGTTTCAGACCCACCTCCCAACCCCGAGGGGACC
CGACAGGCCCGAAGGAATAGAAGAAGAAGGTGGAGAGAGAGACAGAGACAGAT
CCATTCGATTAGTGAACGGATCTCGACGGGATCGATTTTAAAAGAAAAGGGGGG
ATTGGGGGGTACAGTGCAGGGGAAAGAATAGTAGACATAATAGCAACAGACAT
ACAAACTAAAGAATTACAAAAACAAATTACAAAAATTCAAAATTTTATCGATAA
GCTTTGCAAAGATGGATAAAGTTTTAAACAGAGAGGAATCTTTGCAGCTAATGG
ACCTTCTAGGTCTGACCCCGTACGCCTCGAGAGATCTGATCATAATCAGCCATAC
CACATTTGTAGAGGTTTTACTTGCTTTAAAAAACCTCCCACACCTCCCCCTGAACC
TGAAACATAAAATGAATGCAATTGTTGTTGTTAACTTGTTTATTGCAGCTTATAAT
GGTTACAAATAAGGCAATAGCATCACAAATTTCACAAATAAGGCATTTTTTTCAC
TGCATTCTAGTTTTGGTTTGTCCAAACTCATCAATGTATCTTATCATGTCTGGATC
TCAAATCCCTCGGAAGCTGCGCCTGTCTTAGGTTGGAGTGATACATTTTTATCACT
TTTACCCGTCTTTGGATTAGGCAGTAGCTCTGACGGCCCTCCTGTCTTAGGTTAGT
GAAAAATGTCACTCTCTTACCCGTCATTGGCTGTCCAGCTTAGCTCGCAGGGGAG
GTGGTCTGCCTGCAGGTTAGAACAGTCTCTTTTCGTATGAGTGCAGTGAGTGG
ACGCCGCCTTCGACCTGGGCTGAAGAAGTTCTTTTCTCGAACTTCAGTTCGC
CGGAATACATCATTGCCCGCACCTGTGTCAGGCTCTTAGCGCCGATATCCTG
GCATGAATGCTGAATTCCGGCGATCAGGTAAGGCACGAATTTGTGAATACT
GCCCTTATCCTGGACAGCTCCAGACACGCCCTGTGCGACTTTGATCTTGTCT
GCCTCGGAAAAATACCTGTTCTGAGAGGACAGATGCTTATCCATGGCGTCC
AGTGACCCCATGCCCCTATATTTCTTCAGTCTGAACCCATCACTAAAGAAGT
ACTCGCCGGGGGCTTCTGTGGTTGCAGCCAGCAGGCTGCCCATCATCACTG
TGCTTGCCCCCAGAGCCAGGGCTTTTGCGATGTGGCCCACATTCTGAATTCC
CCCGTCAGCGATCACTGGGACTCCGAATCTCCGGGCATACTCGTACACCTT
GTAGACAGCAGTTGCCTGAGGTCGTCCACAGGCCAGCACTTCCTGAATGAT
GCAGATTGATCCACTCCCCATTCCGACCCTCAGAGCATCCACTCCTGCGTCA
ATCAGGTTTTTGGCCTGGGCTGCGGTCACGACATTGCCTCCGATGACCTGC
AGATTTGGGTACTTGTCCTTAATGTACTTGATCATATTAATCTGGAAGATGC
TGTTTCCCTGGCTTGAATCCAGCACGACCACGTCCACCCCTGCCTGAGCCAG
CAGATCCAGGCGATATTTATCGTCCTCGTGTGTGCCAATAGCGGCTCCACAC
AGCAGCTGTTTCTTTGCGTCCTTACTAGCCAGAGGGTAATCTCGATTTTTCT
TCAGGTCGGTGCGGGCAATGATTGCCACCAGCTCATCGTCTTCATTCACGAT
AGGCAGTTTTCCTTTCTTAGACCGCTGCAGAATCTCGTTGGCTTCCTTCAGT
GTGATGCCGGCAGGTGCGACCACCAGATCTTCGCGTTTGGTCATAATCTCTT
CCAGAAAACAGTCATGCTCTTCCTCCTTCAGGAAATCGATGTCTCGACTAGA
AATGATTCCCACCAGTCGGCTGCCCATTCGTCCAGTATCTGTAATGGGGATG
CCGCAAAATCCGTGCCTAGCTTTGGCCTCGAACACATCGCGGACCCTGTCCT
TGGGGCTCAGGACCACTGGGTCGGTGATAAAGCCCTGTTCGTATTTCTTCAC
CTTTCTGACCTCATTGGCCTGAAATTCTGGAGTGCAGTTATGGTGAATGAAC
CCGATCCCGCCTGTCAGTGCCATAGCAATGGCCATGCCAGCCTCGGTGACA
GTGTCCATAGGGGAGCTCACCAGGGGTGTCTTCAGGGTGATTTTCTTGGTC
AGGGCAGAAGTCAGATCCACCTGGTCTGCGGTAAAATCAATATAGCCGGGC
AGGATCAGGAAGTCGTTGTAAGTCAGCCCGTCTCCACAATTAAACAGCTGCT
GGGCGGTCAGTCCATCATCAGGGACATAGGAAGTGCCTCCAGAAATCAGGT
AGTCGGCCATGGTGGCGCTAGCCCTGGGGAGAGAGGTCGGTGATTCGGTCAAC
GAGGGAGCCGACTGCCGACGTGCGCTCCGGAGGCTTGCAGAATGCGGAACACCG
CGCGGGCAGGAACAGGGCCCACACTACCGCCCCACACCCCGCCTCCCGCACCGC
CCCTTCCCGGCCGCTGCTCTCGGCGCGCCCCGCTGAGCAGCCGCTATTGGCCACA
GCCCATCGCGGTCGGCGCGCTGCCATTGCTCCCTGGCGCTGTCCGTCTGCGAGGG
TACTAGTGAGACGTGCGGCTTCCGTTTGTCACGTCCGGCACGCCGCGAACCGCAA
GGAACCTTCCCGACTTAGGGGCGGAGCAGGAAGCGTCGCCGGGGGGCCCACAAG
GGTAGCGGCGAAGATCCGGGTGACGCTGCGAACGGACGTGAAGAATGTGCGAG
ACCCAGGGTCGGCGCCGCTGCGTTTCCCGGAACCACGCCCAGAGCAGCCGCGTC
CCTGCGCAAACCCAGGGCTGCCAAGGAAAAGGCGCAACCCCAACCCCGTGGTTA
ATTAAGGTGAAAGGAGTGGGAATTGGCTCCGGTGCCCGTCAGTGGGCAGAGCGC
ACATCGCCCACAGTCCCCGAGAAGTTGGGGGGAGGGGTCGGCAATTGAA
Italics: co:hGBA transgene
BOLD: reverse complement of IMPDH2(IY) transgene (SEQ ID NO: 10)
SEQ ID NO: 5: codon optimized AGA
>co.hAGA
ATGCAACTTCGAAACCCAGAGCTCCACCTCGGATGTGCCCTTGCTCTGAGGTTCC
TGGCGCTGGTGTCTTGGGATATACCCGGAGCACGCGCTCTGGACAACGGGCTGG
CCCGGACTCCAACCATGGGTTGGCTCCATTGGGAAAGGTTTATGTGCAACTTGGA
CTGCCAGGAAGAACCCGACTCCTGTATTTCCGAGAAACTCTTCATGGAGATGGCC
GAGCTGATGGTTAGCGAAGGCTGGAAGGATGCCGGTTATGAATACTTGTGTATC
GACGATTGTTGGATGGCTCCCCAGCGGGACAGTGAAGGACGACTCCAGGCAGAT
CCGCAACGGTTCCCTCATGGCATACGGCAGCTCGCCAATTACGTGCACAGCAAG
GGTTTGAAGCTGGGGATATATGCTGACGTGGGCAACAAAACCTGTGCTGGTTTCC
CCGGCAGCTTCGGCTACTATGATATAGATGCACAAACCTTCGCTGATTGGGGCGT
GGACCTGCTTAAATTTGACGGCTGTTACTGCGACAGCTTGGAAAACCTCGCCGAT
GGATATAAACACATGAGCCTTGCACTCAATCGGACTGGCCGGAGCATTGTCTACT
CTTGCGAGTGGCCATTGTACATGTGGCCTTTCCAGAAGCCTAACTATACGGAGAT
TAGACAGTATTGTAATCACTGGAGAAACTTTGCAGATATCGACGACTCATGGAA
GTCCATCAAATCTATTCTGGACTGGACTTCATTCAATCAGGAGCGCATCGTCGAT
GTTGCCGGTCCAGGTGGATGGAACGACCCTGACATGCTCGTAATTGGGAATTTCG
GACTGTCCTGGAATCAGCAGGTCACACAGATGGCTTTGTGGGCTATCATGGCAGC
CCCACTCTTTATGTCTAACGATTTGCGGCATATTTCACCACAGGCCAAAGCCCTG
CTGCAAGATAAGGACGTCATAGCGATTAACCAGGACCCACTGGGAAAGCAGGGC
TACCAGCTGAGACAGGGCGACAATTTTGAGGTCTGGGAAAGACCTCTTAGCGGG
CTGGCGTGGGCCGTAGCCATGATTAATCGCCAGGAAATTGGCGGCCCTCGCTCTT
ACACTATCGCGGTCGCCAGTCTGGGCAAGGGAGTCGCTTGTAACCCCGCCTGCTT
CATAACTCAGTTGCTGCCCGTGAAACGGAAGCTGGGCTTCTATGAATGGACTAGC
AGACTCCGCAGTCATATTAATCCGACTGGTACGGTGCTGCTGCAACTGGAGAATA
CCATGCAGATGTCACTTAAGGATCTTCTGTGA
SEQ ID NO: 6, GBA transgene
>co.hGBA
ATGGAGTTCTCAAGCCCCTCTCGGGAAGAATGCCCAAAACCTCTGTCACGGGTGT
CTATCATGGCTGGATCACTGACTGGCCTGCTGCTGCTGCAGGCCGTGAGCTGGGC
CTCCGGAGCCCGGCCTTGCATCCCAAAGTCTTTCGGCTACAGCTCCGTGGTGTGC
GTGTGCAACGCCACCTATTGTGACTCCTTCGATCCCCCTACCTTTCCCGCCCTGGG
CACATTTTCTCGGTACGAGTCTACACGCAGCGGCAGGAGAATGGAGCTGAGCAT
GGGCCCTATCCAGGCCAATCACACCGGAACAGGCCTGCTGCTGACCCTGCAGCC
AGAGCAGAAGTTCCAGAAGGTGAAGGGCTTTGGAGGAGCAATGACAGACGCAG
CCGCCCTGAACATCCTGGCCCTGTCCCCACCCGCCCAGAATCTGCTGCTGAAGTC
CTACTTCTCTGAGGAGGGCATCGGCTATAACATCATCAGGGTGCCCATGGCCAGC
TGCGACTTTTCCATCAGAACCTACACATATGCCGATACCCCTGACGATTTCCAGC
TGCACAATTTTTCCCTGCCAGAGGAGGATACAAAGCTGAAGATCCCACTGATCCA
CAGGGCCCTGCAGCTGGCCCAGAGGCCCGTGAGCCTGCTGGCCAGCCCCTGGAC
CTCCCCTACATGGCTGAAGACCAACGGCGCCGTGAATGGCAAGGGCTCTCTGAA
GGGACAGCCAGGCGACATCTACCACCAGACATGGGCCCGCTATTTCGTGAAGTTT
CTGGATGCCTACGCCGAGCACAAGCTGCAGTTCTGGGCCGTGACCGCAGAGAAC
GAGCCTTCTGCCGGCCTGCTGAGCGGCTATCCCTTCCAGTGCCTGGGCTTTACAC
CTGAGCACCAGAGGGACTTTATCGCCAGAGATCTGGGCCCAACCCTGGCCAACT
CCACACACCACAATGTGCGGCTGCTGATGCTGGACGATCAGCGCCTGCTGCTGCC
TCACTGGGCCAAGGTGGTGCTGACCGACCCAGAGGCCGCCAAGTACGTGCACGG
CATCGCCGTGCACTGGTATCTGGATTTCCTGGCACCAGCAAAGGCCACCCTGGGA
GAGACACACAGGCTGTTCCCTAACACCATGCTGTTTGCCAGCGAGGCCTGCGTGG
GCTCCAAGTTTTGGGAGCAGTCCGTGCGGCTGGGCTCTTGGGACAGGGGCATGC
AGTACTCCCACTCTATCATCACCAATCTGCTGTATCACGTGGTGGGCTGGACAGA
CTGGAACCTGGCCCTGAATCCAGAGGGCGGCCCCAACTGGGTGAGAAATTTCGT
GGATAGCCCCATCATCGTGGACATCACCAAGGATACATTCTACAAGCAGCCAAT
GTTTTATCACCTGGGCCACTTCTCTAAGTTTATCCCAGAGGGCAGCCAGAGGGTG
GGCCTGGTGGCCAGCCAGAAGAACGACCTGGATGCAGTGGCCCTGATGCACCCT
GACGGCTCCGCCGTGGTGGTGGTGCTGAATCGCTCTAGCAAGGACGTGCCTCTGA
CCATCAAGGACCCCGCCGTGGGCTTTCTGGAGACCATTTCACCCGGCTATTCTAT
TCATACCTATCTGTGGAGGAGGCAGTAA
SEQ ID NO: 7: coIMPDH2(IY) transgene
ATGGCCGACTACCTGATTTCTGGAGGCACTTCCTATGTCCCTGATGATGGACTGA
CCGCCCAGCAGCTGTTTAATTGTGGAGACGGGCTGACTTACAACGACTTCCTGAT
CCTGCCCGGCTATATTGATTTTACCGCAGACCAGGTGGATCTGACTTCTGCCCTG
ACCAAGAAAATCACCCTGAAGACACCCCTGGTGAGCTCCCCTATGGACACTGTC
ACCGAGGCTGGCATGGCCATTGCTATGGCACTGACAGGCGGGATCGGGTTCATTC
ACCATAACTGCACTCCAGAATTTCAGGCCAATGAGGTCAGAAAGGTGAAGAAAT
ACGAACAGGGCTTTATCACCGACCCAGTGGTCCTGAGCCCCAAGGACAGGGTCC
GCGATGTGTTCGAGGCCAAAGCTAGGCACGGATTTTGCGGCATCCCCATTACAGA
TACTGGACGAATGGGCAGCCGACTGGTGGGAATCATTTCTAGTCGAGACATCGA
TTTCCTGAAGGAGGAAGAGCATGACTGTTTTCTGGAAGAGATTATGACCAAACG
CGAAGATCTGGTGGTCGCACCTGCCGGCATCACACTGAAGGAAGCCAACGAGAT
TCTGCAGCGGTCTAAGAAAGGAAAACTGCCTATCGTGAATGAAGACGATGAGCT
GGTGGCAATCATTGCCCGCACCGACCTGAAGAAAAATCGAGATTACCCTCTGGCT
AGTAAGGACGCAAAGAAACAGCTGCTGTGTGGAGCCGCTATTGGCACACACGAG
GACGATAAATATCGCCTGGATCTGCTGGCTCAGGCAGGGGTGGACGTGGTCGTG
CTGGATTCAAGCCAGGGAAACAGCATCTTCCAGATTAATATGATCAAGTACATTA
AGGACAAGTACCCAAATCTGCAGGTCATCGGAGGCAATGTCGTGACCGCAGCCC
AGGCCAAAAACCTGATTGACGCAGGAGTGGATGCTCTGAGGGTCGGAATGGGGA
GTGGATCAATCTGCATCATTCAGGAAGTGCTGGCCTGTGGACGACCTCAGGCAAC
TGCTGTCTACAAGGTGTACGAGTATGCCCGGAGATTCGGAGTCCCAGTGATCGCT
GACGGGGGAATTCAGAATGTGGGCCACATCGCAAAAGCCCTGGCTCTGGGGGCA
AGCACAGTGATGATGGGCAGCCTGCTGGCTGCAACCACAGAAGCCCCCGGCGAG
TACTTCTTTAGTGATGGGTTCAGACTGAAGAAATATAGGGGCATGGGGTCACTGG
ACGCCATGGATAAGCATCTGTCCTCTCAGAACAGGTATTTTTCCGAGGCAGACAA
GATCAAAGTCGCACAGGGCGTGTCTGGAGCTGTCCAGGATAAGGGCAGTATTCA
CAAATTCGTGCCTTACCTGATCGCCGGAATTCAGCATTCATGCCAGGATATCGGC
GCTAAGAGCCTGACACAGGTGCGGGCAATGATGTATTCCGGCGAACTGAAGTTC
GAGAAAAGAACTTCTTCAGCCCAGGTCGAAGGCGGCGTCCACTCACTGCACTCA
TACGAAAAGAGACTGTTCTAA
SEQ ID NO: 8 ASAH1 transgene
>hASAH1
ATGCCGGGCCGGAGTTGCGTCGCCTTAGTCCTCCTGGCTGCCGCCGTCAGCTGTGCCGTCGCG
CAGCACGCGCCGCCGTGGACAGAGGACTGCAGAAAATCAACCTATCCTCCTTCAGGACCAACG
TACAGAGGTGCAGTTCCATGGTACACCATAAATCTTGACTTACCACCCTACAAAAGATGGCA
TGAATTGATGCTTGACAAGGCACCAGTGCTAAAGGTTATAGTGAATTCTCTGAAGAATATGA
TAAATACATTCGTGCCAAGTGGAAAAATTATGCAGGTGGTGGATGAAAAATTGCCTGGCCTA
CTTGGCAACTTTCCTGGCCCTTTTGAAGAGGAAATGAAGGGTATTGCCGCTGTTACTGATAT
ACCTTTAGGAGAGATTATTTCATTCAATATTTTTTATGAATTATTTACCATTTGTACTTCAA
TAGTAGCAGAAGACAAAAAAGGTCATCTAATACATGGGAGAAACATGGATTTTGGAGTATTT
CTTGGGTGGAACATAAATAATGATACCTGGGTCATAACTGAGCAACTAAAACCTTTAACAGT
GAATTTGGATTTCCAAAGAAACAACAAAACTGTCTTCAAGGCTTCAAGCTTTGCTGGCTATG
TGGGCATGTTAACAGGATTCAAACCAGGACTGTTCAGTCTTACACTGAATGAACGTTTCAGT
ATAAATGGTGGTTATCTGGGTATTCTAGAATGGATTCTGGGAAAGAAAGATGTCATGTGGAT
AGGGTTCCTCACTAGAACAGTTCTGGAAAATAGCACAAGTTATGAAGAAGCCAAGAATTTAT
TGACCAAGACCAAGATATTGGCCCCAGCCTACTTTATCCTGGGAGGCAACCAGTCTGGGGAA
GGTTGTGTGATTACACGAGACAGAAAGGAATCATTGGATGTATATGAACTCGATGCTAAGCA
GGGTAGATGGTATGTGGTACAAACAAATTATGACCGTTGGAAACATCCCTTCTTCCTTGATG
ATCGCAGAACGCCTGCAAAGATGTGTCTGAACCGCACCAGCCAAGAGAATATCTCATTTGAA
ACCATGTATGATGTCCTGTCAACAAAACCTGTCCTCAACAAGCTGACCGTATACACAACCTTG
ATAGATGTTACCAAAGGTCAATTCGAAACTTACCTGCGGGACTGCCCTGACCCTTGTATAGG
TTGGTGA
SEQ ID NO: 9 (GAA transgene)
>co.hGAA
ATGGGCGTGAGGCACCCCCCTTGCTCTCACAGGCTGCTGGCCGTGTGCGCACTGGTGAGCCTG
GCCACCGCCGCCCTGCTGGGCCACATCCTGCTGCACGACTTCCTGCTGGTGCCCAGGGAGCTG
TCCGGCAGCTCCCCAGTGCTGGAGGAGACCCACCCAGCACACCAGCAGGGCGCCTCTCGGCCA
GGCCCCCGCGATGCACAGGCACACCCAGGCCGGCCCCGCGCCGTGCCAACCCAGTGCGACGTG
CCACCCAACAGCCGGTTTGACTGTGCCCCCGATAAGGCCATCACACAGGAGCAGTGCGAGGCC
AGGGGCTGCTGTTATATCCCTGCAAAGCAGGGCCTCCAGGGCGCCCAGATGGGACAGCCATGG
TGTTTCTTTCCTCCATCTTACCCCAGCTATAAGCTGGAGAATCTGTCTAGCTCCGAGATGGGC
TACACAGCCACCCTGACAAGAACCACACCAACATTCTTTCCCAAGGACATCCTGACCCTGCGG
CTGGACGTGATGATGGAGACAGAGAACCGCCTGCACTTCACCATCAAGGACCCCGCCAATAG
GAGATATGAGGTGCCTCTGGAGACCCCACACGTGCACTCTCGGGCCCCTAGCCCACTGTACTC
CGTGGAGTTCTCTGAGGAGCCATTTGGCGTGATCGTGCGGCGCCAGCTGGATGGACGCGTGCT
GCTGAACACCACAGTGGCCCCCCTGTTCTTTGCCGACCAGTTCCTCCAGCTGAGCACATCCCT
GCCCTCCCAGTATATCACCGGCCTGGCCGAGCACCTGTCTCCTCTGATGCTGTCTACCAGCTG
GACAAGGATCACCCTGTGGAACAGAGACCTGGCACCAACCCCTGGCGCAAATCTGTACGGCAG
CCACCCTTTCTATCTGGCCCTGGAGGATGGAGGCTCCGCCCACGGCGTGTTTCTGCTGAACTC
TAATGCCATGGACGTGGTGCTCCAGCCAAGCCCCGCCCTGTCCTGGCGGTCTACCGGCGGCAT
CCTGGACGTGTACATCTTCCTGGGCCCTGAGCCAAAGTCCGTGGTGCAGCAGTACCTGGACGT
GGTGGGCTATCCTTTCATGCCCCCTTACTGGGGACTGGGATTTCACCTGTGCCGCTGGGGCTA
TTCTAGCACAGCCATCACCCGGCAGGTGGTGGAGAACATGACCCGCGCCCACTTTCCACTGGA
TGTGCAGTGGAATGACCTGGATTACATGGACTCCAGGAGAGACTTCACCTTCAACAAGGACG
GCTTCAGGGATTTTCCCGCCATGGTGCAGGAGCTGCACCAGGGCGGCCGGCGCTACATGATGA
TCGTGGACCCCGCCATCTCCTCTAGCGGACCTGCCGGCAGCTACAGACCATATGACGAGGGCC
TGAGGAGAGGCGTGTTCATCACAAACGAGACCGGCCAGCCTCTGATCGGCAAGGTCTGGCCA
GGCTCCACCGCCTTCCCAGACTTCACCAATCCAACCGCCCTGGCCTGGTGGGAGGACATGGTG
GCCGAGTTCCACGACCAGGTGCCTTTTGATGGCATGTGGATCGACATGAACGAGCCATCTAA
TTTCATCAGGGGCAGCGAGGACGGCTGCCCCAACAATGAGCTGGAGAACCCACCATATGTGCC
TGGCGTGGTGGGAGGCACCCTCCAGGCAGCAACCATCTGTGCCTCCTCTCACCAGTTTCTGTC
TACACACTATAACCTGCACAATCTGTACGGACTGACCGAGGCAATCGCCAGCCACAGAGCCCT
GGTGAAGGCCAGGGGCACAAGACCTTTCGTGATCTCCAGGTCTACCTTTGCCGGACACGGCAG
ATACGCAGGACACTGGACCGGCGACGTGTGGAGCAGCTGGGAGCAGCTGGCCTCTAGCGTGCC
AGAGATCCTCCAGTTCAACCTGCTGGGCGTGCCCCTGGTGGGAGCAGACGTGTGCGGCTTTCT
GGGCAATACATCCGAGGAGCTGTGCGTGAGGTGGACCCAGCTGGGAGCCTTCTATCCCTTCAT
GCGCAACCACAATAGCCTGCTGTCCCTGCCTCAGGAGCCATACAGCTTCTCCGAGCCTGCACA
GCAGGCAATGAGGAAGGCCCTGACACTGCGCTATGCCCTGCTGCCACACCTGTACACCCTGTT
TCACCAGGCACACGTGGCAGGAGAGACAGTGGCCCGGCCCCTGTTCCTGGAGTTTCCTAAGGA
TTCCTCTACCTGGACAGTGGACCACCAGCTGCTGTGGGGAGAGGCCCTGCTGATCACCCCCGT
GCTCCAGGCAGGCAAGGCAGAGGTGACAGGCTATTTCCCTCTGGGCACATGGTACGACCTCCA
GACCGTGCCAGTGGAGGCCCTGGGCAGCCTGCCTCCACCACCTGCCGCCCCCCGCGAGCCTGCC
ATCCACTCCGAGGGACAGTGGGTGACACTGCCAGCACCTCTGGACACCATCAACGTGCACCTG
AGGGCCGGCTATATCATCCCCCTCCAGGGCCCTGGCCTGACCACAACCGAGTCCAGACAGCAG
CCAATGGCCCTGGCCGTGGCCCTGACCAAGGGAGGCGAGGCCAGGGGCGAGCTGTTCTGGGAC
GATGGCGAGTCTCTGGAGGTGCTGGAGAGAGGCGCCTACACACAGGTCATCTTCCTGGCCAG
GAACAATACAATCGTGAATGAGCTGGTGAGAGTGACCTCTGAGGGAGCAGGACTCCAGCTCC
AGAAGGTGACAGTGCTGGGAGTGGCAACCGCACCACAGCAGGTGCTGAGCAACGGCGTGCCC
GTGAGCAATTTCACATACTCCCCTGATACCAAGGTGCTGGACATCTGCGTGAGCCTGCTGATG
GGCGAGCAGTTTCTGGTGTCCTGGTGTTGA

We claim:

1. A dual promoter lentivirus vector that expresses a protein of interest encoded by a transgene and a mutant form of inosine-5′-monophosphate dehydrogenase 2 (IMPDH2(IY)) when transduced into a host cell.

2. The dual promoter lentivirus vector of claim 1, wherein the vector when transduced into the host cell confers resistance to mycophenolic acid (MPA) and/or mycophenolate mofetil (MMF) in vitro and/or in vivo.

3. The dual promoter lentivirus vector of any one of the preceding claims, wherein the host cell is a hematopoietic cell.

4. The dual promoter lentivirus vector of any one of the preceding claims, wherein a first promoter is hPGK promoter and a second promoter is EF1α promoter.

5. The dual promoter lentivirus vector of any one of the preceding claims, wherein the mutant form of inosine-5′-monophosphate dehydrogenase 2 (IMPDH2) contains T333I and S351Y mutations in the IMPDH2 transgene.

6. The dual promoter lentivirus vector of any one of the preceding claims, wherein the protein of interest is a protein or an enzyme associated with a lysosomal storage disorder.

7. The dual promoter lentivirus vector of claim 6, wherein the protein of interest is α-galactosidase A and the lysosomal storage disorder is Fabry disease.

8. The dual promoter lentivirus vector of claim 7, wherein α-galactosidase A is encoded by the transgene AGA of SEQ ID NO:5 or a DNA sequence with 80% identity to SEQ ID NO:5.

9. The dual promoter lentivirus vector of claim 7, wherein the vector is encoded by the DNA sequence of SEQ ID NO: 3 or a DNA sequence with 80% identity to SEQ ID NO: 3.

10. The dual promoter lentivirus vector of any one of claims 1-6, wherein the protein of interest is glucocerebrosidase (GBA protein).

11. The dual promoter lentivirus vector of claim 10, wherein glucocerebrosidase is encoded by the transgene GBA of SEQ ID NO:6 or a DNA sequence with 80% identity to SEQ ID NO:6.

12. The dual promoter lentivirus vector of any one of claims 10-11, wherein the vector is encoded by the DNA sequence of SEQ ID NO:4 or a DNA sequence with 80% identity to SEQ ID NO:4.

13. The dual promoter lentivirus vector of any one of claims 1-6, wherein the protein of interest is ASAH1.

14. The dual promoter lentivirus vector of claim 13, wherein ASAHI is encoded by the transgene of SEQ ID NO:8 or a DNA sequence with 80% identity to SEQ ID NO:8.

15. The dual promoter lentivirus vector of any one of claims 1-6, wherein the protein of interest is GAA.

16. The dual promoter lentivirus vector of claim 15, wherein GAA is encoded by the transgene of SEQ ID NO:9 or a DNA sequence with 80% identity to SEQ ID NO:9.

17. The dual promoter lentivirus vector of any one of claims 10-16, wherein the vector confers resistance to mycophenolic acid (MPA) and mycophenolate mofetil (MMF) in vitro and/or in vivo when expressed within the host cell.

18. A host cell expressing the dual promoter lentivirus vector of any one of the preceding claims.

19. The host cell of claim 18, wherein the host cell is a hematopoietic cell, preferably a hematopoietic stem cell.

20. A method of treating a subject having a disease or disorder associated with a defect in a single protein, the method comprising the steps of
i) obtaining hematopoietic stem cells (HSCs) from the subject and/or HSCs from a suitable donor,
ii) transducing the HSCs in vitro or ex vivo with a dual promoter lentivirus vector that expresses a functional form of the protein of interest and a mutant form of inosine-5′-monophosphate dehydrogenase 2 (IMPDH2(IY)) encoded by SEQ ID NO:7 in the transduced cells,
iii) introducing the transduced HSCs into the subject, and
iv) administering to the subject an amount of mycophenolate mofetil (MMF) sufficient to enrich the population of lentivirus vector transduced HSCs engrafted in the subject, wherein the method treats one or more symptoms of the disease or disorder.

21. The method of claim 20, wherein the subject has a lysosomal storage disorder (LSD), and wherein the protein of interest is associated with the LSD.

22. The method of claim 20 or 21, wherein the dual promoter lentivirus vector in step (ii) is the dual promoter lentivirus vector of any one of claims 1-17.

23. The method of any one of claims 20-22, wherein the subject is in need of treatment for Fabry disease, and wherein the dual promoter lentivirus vector of step (ii) expresses α-galactosidase A and IMPDH2(IY) in the transduced cells, and wherein step (iv) comprises administering to the subject an amount of mycophenolate mofetil (MMF) sufficient to enrich the population of lentivirus vector transduced hematopoietic cells in the subject and treat one or more symptoms of Fabry disease.

24. The method of claim 20, wherein the dual promoter lentiviral vector comprises SEQ ID NO:3 or a DNA sequence having at least 80% sequence similarity to SEQ ID NO:3 for the treatment of Fabry disease.

25. The method of claim 20-22, wherein the dual promoter lentivirus vector of step (ii) expresses glucocerebrosidase and IMPDH2(IY) in the transduced cells, wherein the method treats one or more symptoms of Gaucher disease.

26. The method of claim 25, wherein the vector comprises SEQ ID NO:4 or a DNA sequence having at least 80% similarity to SEQ ID NO:4.

27. The method of any one of claims 20-22, wherein the subject is in need of treatment for Farber disease, and wherein the dual promoter lentivirus vector of step (ii) expresses ASAH1 and IMPDH2(IY) in the transduced cells, and wherein step (iv) comprises administering to the subject an amount of mycophenolate mofetil (MMF) sufficient to enrich the population of lentivirus vector transduced hematopoietic cells in the subject and treat one or more symptoms of Farber disease.

28. The method of any one of claims 20-22, wherein the subject is in need of treatment for Pompe disease, and wherein the dual promoter lentivirus vector of step (ii) expresses GAA and IMPDH2(IY) in the transduced cells, and wherein step (iv) comprises administering to the subject an amount of mycophenolate mofetil (MMF) sufficient to enrich the population of lentivirus vector transduced hematopoietic cells in the subject and treat one or more symptoms of Pompe disease.

29. The method of any one of claims 20-28, wherein the subject is human.

30. The method of any one of claims 20-29, wherein the transduced HSCs are injected into the patient.

31. The method of any one of claims 20-30, wherein the MMF is administered in an amount of about 0.1-5 mg/kg three times a day.

32. The method of any one of claims 20-31, wherein the MMF is administered at 5-10 mg/kg three times a day or 1000 mg twice a day.

33. Use of the dual promoter lentivirus vector of any one of claims 1-17 or the host cell of claim 18-19 for the treatment of a disease or disorder.

34. The dual promoter lentivirus vector of any one of claims 1-5, wherein the protein of interest is a protein or an enzyme associated with a hematopoietic disease.

35. The dual promoter lentivirus vector of claim 34, wherein the hematopoietic disease is a blood clotting disorder.

36. The dual promoter lentivirus vector of claim 35, wherein the blood clotting disorder is hemophilia.

37. The dual promoter lentivirus vector of claim 35, wherein the transgene of interest is Factor VIII (SEQ ID NO:11) or a DNA sequence with at least 80% identity to SEQ ID NO: 11.

38. The method of claim 20, wherein the subject has a hematopoietic disease, and wherein the protein of interest is associated with the hematopoietic disease.

39. The method of claim 38, wherein the hematopoietic disease is a blood clotting disease.

40. The method of claim 39, wherein the blood clotting disease is hemophilia and the protein of interest is Factor VIII encoded by the transgene of SEQ ID NO:11 or a DNA sequence with at least 8% T identity to SEQ ID NO:11.


SEQUENCE LISTING (12 sequences)

SEQ ID NO: 1 - DNA, 8523 bp, synthetic construct
  source 1..8523: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggcggatggcgttaacatatgacaactttctcccgggtaatctgaccgttcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 2 - DNA, 10025 bp, synthetic construct
  source 1..10025: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 3 - DNA, 11298 bp, synthetic construct
  source 1..11298: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgccaccatgcaacttcgaaacccagagctccacctcggatgtgcccttgctctgaggttcctggcgctggtgtcttgggatatacccggagcacgcgctctggacaacgggctggcccggactccaaccatgggttggctccattgggaaaggtttatgtgcaacttggactgccaggaagaacccgactcctgtatttccgagaaactcttcatggagatggccgagctgatggttagcgaaggctggaaggatgccggttatgaatacttgtgtatcgacgattgttggatggctccccagcgggacagtgaaggacgactccaggcagatccgcaacggttccctcatggcatacggcagctcgccaattacgtgcacagcaagggtttgaagctggggatatatgctgacgtgggcaacaaaacctgtgctggtttccccggcagcttcggctactatgatatagatgcacaaaccttcgctgattggggcgtggacctgcttaaatttgacggctgttactgcgacagcttggaaaacctcgccgatggatataaacacatgagccttgcactcaatcggactggccggagcattgtctactcttgcgagtggccattgtacatgtggcctttccagaagcctaactatacggagattagacagtattgtaatcactggagaaactttgcagatatcgacgactcatggaagtccatcaaatctattctggactggacttcattcaatcaggagcgcatcgtcgatgttgccggtccaggtggatggaacgaccctgacatgctcgtaattgggaatttcggactgtcctggaatcagcaggtcacacagatggctttgtgggctatcatggcagccccactctttatgtctaacgatttgcggcatatttcaccacaggccaaagccctgctgcaagataaggacgtcatagcgattaaccaggacccactgggaaagcagggctaccagctgagacagggcgacaattttgaggtctgggaaagacctcttagcgggctggcgtgggccgtagccatgattaatcgccaggaaattggcggccctcgctcttacactatcgcggtcgccagtctgggcaagggagtcgcttgtaaccccgcctgcttcataactcagttgctgcccgtgaaacggaagctgggcttctatgaatggactagcagactccgcagtcatattaatccgactggtacggtgctgctgcaactggagaataccatgcagatgtcacttaaggatcttctgtgagaacccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcagg

SEQ ID NO: 4 - DNA, 11626 bp, synthetic construct
  source 1..11626: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgctagcgccaccatggagttctcaagcccctctcgggaagaatgcccaaaacctctgtcacgggtgtctatcatggctggatcactgactggcctgctgctgctgcaggccgtgagctgggcctccggagcccggccttgcatcccaaagtctttcggctacagctccgtggtgtgcgtgtgcaacgccacctattgtgactccttcgatccccctacctttcccgccctgggcacattttctcggtacgagtctacacgcagcggcaggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatcagggtgcccatggccagctgcgacttttccatcagaacctacacatatgccgatacccctgacgatttccagctgcacaatttttccctgccagaggaggatacaaagctgaagatcccactgatccacagggccctgcagctggcccagaggcccgtgagcctgctggccagcccctggacctcccctacatggctgaagaccaacggcgccgtgaatggcaagggctctctgaagggacagccaggcgacatctaccaccagacatgggcccgctatttcgtgaagtttctggatgcctacgccgagcacaagctgcagttctgggccgtgaccgcagagaacgagccttctgccggcctgctgagcggctatcccttccagtgcctgggctttacacctgagcaccagagggactttatcgccagagatctgggcccaaccctggccaactccacacaccacaatgtgcggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacacaggctgttccctaacaccatgctgtttgccagcgaggcctgcgtgggctccaagttttgggagcagtccgtgcggctgggctcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgcagtggccctgatgcaccctgacggctccgccgtggtggtggtgctgaatcgctctagcaaggacgtgcctctgaccatcaaggaccccgccgtgggctttctggagaccatttcacccggctattctattcatacctatctgtggaggaggcagtaacctgcaggggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaa

SEQ ID NO: 5 - DNA, 1290 bp, synthetic construct
  source 1..1290: mol_type = other DNA
atgcaacttcgaaacccagagctccacctcggatgtgcccttgctctgaggttcctggcgctggtgtcttgggatatacccggagcacgcgctctggacaacgggctggcccggactccaaccatgggttggctccattgggaaaggtttatgtgcaacttggactgccaggaagaacccgactcctgtatttccgagaaactcttcatggagatggccgagctgatggttagcgaaggctggaaggatgccggttatgaatacttgtgtatcgacgattgttggatggctccccagcgggacagtgaaggacgactccaggcagatccgcaacggttccctcatggcatacggcagctcgccaattacgtgcacagcaagggtttgaagctggggatatatgctgacgtgggcaacaaaacctgtgctggtttccccggcagcttcggctactatgatatagatgcacaaaccttcgctgattggggcgtggacctgcttaaatttgacggctgttactgcgacagcttggaaaacctcgccgatggatataaacacatgagccttgcactcaatcggactggccggagcattgtctactcttgcgagtggccattgtacatgtggcctttccagaagcctaactatacggagattagacagtattgtaatcactggagaaactttgcagatatcgacgactcatggaagtccatcaaatctattctggactggacttcattcaatcaggagcgcatcgtcgatgttgccggtccaggtggatggaacgaccctgacatgctcgtaattgggaatttcggactgtcctggaatcagcaggtcacacagatggctttgtgggctatcatggcagccccactctttatgtctaacgatttgcggcatatttcaccacaggccaaagccctgctgcaagataaggacgtcatagcgattaaccaggacccactgggaaagcagggctaccagctgagacagggcgacaattttgaggtctgggaaagacctcttagcgggctggcgtgggccgtagccatgattaatcgccaggaaattggcggccctcgctcttacactatcgcggtcgccagtctgggcaagggagtcgcttgtaaccccgcctgcttcataactcagttgctgcccgtgaaacggaagctgggcttctatgaatggactagcagactccgcagtcatattaatccgactggtacggtgctgctgcaactggagaataccatgcagatgtcacttaaggatcttctgtga

SEQ ID NO: 6 - DNA, 1611 bp, synthetic construct
  source 1..1611: mol_type = other DNA
atggagttctcaagcccctctcgggaagaatgcccaaaacctctgtcacgggtgtctatcatggctggatcactgactggcctgctgctgctgcaggccgtgagctgggcctccggagcccggccttgcatcccaaagtctttcggctacagctccgtggtgtgcgtgtgcaacgccacctattgtgactccttcgatccccctacctttcccgccctgggcacattttctcggtacgagtctacacgcagcggcaggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatcagggtgcccatggccagctgcgacttttccatcagaacctacacatatgccgatacccctgacgatttccagctgcacaatttttccctgccagaggaggatacaaagctgaagatcccactgatccacagggccctgcagctggcccagaggcccgtgagcctgctggccagcccctggacctcccctacatggctgaagaccaacggcgccgtgaatggcaagggctctctgaagggacagccaggcgacatctaccaccagacatgggcccgctatttcgtgaagtttctggatgcctacgccgagcacaagctgcagttctgggccgtgaccgcagagaacgagccttctgccggcctgctgagcggctatcccttccagtgcctgggctttacacctgagcaccagagggactttatcgccagagatctgggcccaaccctggccaactccacacaccacaatgtgcggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacacaggctgttccctaacaccatgctgtttgccagcgaggcctgcgtgggctccaagttttgggagcagtccgtgcggctgggctcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgcagtggccctgatgcaccctgacggctccgccgtggtggtggtgctgaatcgctctagcaaggacgtgcctctgaccatcaaggaccccgccgtgggctttctggagaccatttcacccggctattctattcatacctatctgtggaggaggcagtaa

SEQ ID NO: 7 - DNA, 1545 bp, synthetic construct
  source 1..1545: mol_type = other DNA
atggccgactacctgatttctggaggcacttcctatgtccctgatgatggactgaccgcccagcagctgtttaattgtggagacgggctgacttacaacgacttcctgatcctgcccggctatattgattttaccgcagaccaggtggatctgacttctgccctgaccaagaaaatcaccctgaagacacccctggtgagctcccctatggacactgtcaccgaggctggcatggccattgctatggcactgacaggcgggatcgggttcattcaccataactgcactccagaatttcaggccaatgaggtcagaaaggtgaagaaatacgaacagggctttatcaccgacccagtggtcctgagccccaaggacagggtccgcgatgtgttcgaggccaaagctaggcacggattttgcggcatccccattacagatactggacgaatgggcagccgactggtgggaatcatttctagtcgagacatcgatttcctgaaggaggaagagcatgactgttttctggaagagattatgaccaaacgcgaagatctggtggtcgcacctgccggcatcacactgaaggaagccaacgagattctgcagcggtctaagaaaggaaaactgcctatcgtgaatgaagacgatgagctggtggcaatcattgcccgcaccgacctgaagaaaaatcgagattaccctctggctagtaaggacgcaaagaaacagctgctgtgtggagccgctattggcacacacgaggacgataaatatcgcctggatctgctggctcaggcaggggtggacgtggtcgtgctggattcaagccagggaaacagcatcttccagattaatatgatcaagtacattaaggacaagtacccaaatctgcaggtcatcggaggcaatgtcgtgaccgcagcccaggccaaaaacctgattgacgcaggagtggatgctctgagggtcggaatggggagtggatcaatctgcatcattcaggaagtgctggcctgtggacgacctcaggcaactgctgtctacaaggtgtacgagtatgcccggagattcggagtcccagtgatcgctgacgggggaattcagaatgtgggccacatcgcaaaagccctggctctgggggcaagcacagtgatgatgggcagcctgctggctgcaaccacagaagcccccggcgagtacttctttagtgatgggttcagactgaagaaatataggggcatggggtcactggacgccatggataagcatctgtcctctcagaacaggtatttttccgaggcagacaagatcaaagtcgcacagggcgtgtctggagctgtccaggataagggcagtattcacaaattcgtgccttacctgatcgccggaattcagcattcatgccaggatatcggcgctaagagcctgacacaggtgcgggcaatgatgtattccggcgaactgaagttcgagaaaagaacttcttcagcccaggtcgaaggcggcgtccactcactgcactcatacgaaaagagactgttctaa

SEQ ID NO: 8 - DNA, 1188 bp, synthetic construct
  source 1..1188: mol_type = other DNA
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 9 - DNA, 2859 bp, synthetic construct
  source 1..2859: mol_type = other DNA
atgggcgtgaggcaccccccttgctctcacaggctgctggccgtgtgcgcactggtgagcctggccaccgccgccctgctgggccacatcctgctgcacgacttcctgctggtgcccagggagctgtccggcagctccccagtgctggaggagacccacccagcacaccagcagggcgcctctcggccaggcccccgcgatgcacaggcacacccaggccggccccgcgccgtgccaacccagtgcgacgtgccacccaacagccggtttgactgtgcccccgataaggccatcacacaggagcagtgcgaggccaggggctgctgttatatccctgcaaagcagggcctccagggcgcccagatgggacagccatggtgtttctttcctccatcttaccccagctataagctggagaatctgtctagctccgagatgggctacacagccaccctgacaagaaccacaccaacattctttcccaaggacatcctgaccctgcggctggacgtgatgatggagacagagaaccgcctgcacttcaccatcaaggaccccgccaataggagatatgaggtgcctctggagaccccacacgtgcactctcgggcccctagcccactgtactccgtggagttctctgaggagccatttggcgtgatcgtgcggcgccagctggatggacgcgtgctgctgaacaccacagtggcccccctgttctttgccgaccagttcctccagctgagcacatccctgccctcccagtatatcaccggcctggccgagcacctgtctcctctgatgctgtctaccagctggacaaggatcaccctgtggaacagagacctggcaccaacccctggcgcaaatctgtacggcagccaccctttctatctggccctggaggatggaggctccgcccacggcgtgtttctgctgaactctaatgccatggacgtggtgctccagccaagccccgccctgtcctggcggtctaccggcggcatcctggacgtgtacatcttcctgggccctgagccaaagtccgtggtgcagcagtacctggacgtggtgggctatcctttcatgcccccttactggggactgggatttcacctgtgccgctggggctattctagcacagccatcacccggcaggtggtggagaacatgacccgcgcccactttccactggatgtgcagtggaatgacctggattacatggactccaggagagacttcaccttcaacaaggacggcttcagggattttcccgccatggtgcaggagctgcaccagggcggccggcgctacatgatgatcgtggaccccgccatctcctctagcggacctgccggcagctacagaccatatgacgagggcctgaggagaggcgtgttcatcacaaacgagaccggccagcctctgatcggcaaggtctggccaggctccaccgccttcccagacttcaccaatccaaccgccctggcctggtgggaggacatggtggccgagttccacgaccaggtgccttttgatggcatgtggatcgacatgaacgagccatctaatttcatcaggggcagcgaggacggctgccccaacaatgagctggagaacccaccatatgtgcctggcgtggtgggaggcaccctccaggcagcaaccatctgtgcctcctctcaccagtttctgtctacacactataacctgcacaatctgtacggactgaccgaggcaatcgccagccacagagccctggtgaaggccaggggcacaagacctttcgtgatctccaggtctacctttgccggacacggcagatacgcaggacactggaccggcgacgtgtggagcagctgggagcagctggcctctagcgtgccagagatcctccagttcaacctgctgggcgtgcccctggtgggagcagacgtgtgcggctttctgggcaatacatccgaggagctgtgcgtgaggtggacccagctgggagccttctatcccttcatgcgcaaccacaatagcctgctgtccctgcctcaggagccatacagcttctccgagcctgcacagcaggcaatgaggaaggccctgacactgcgctatgccctgctgccacacctgtacaccctgtttcaccaggcacacgtggcaggagagacagtggcccggcccctgttcctggagtttcctaaggattcctctacctggacagtggaccaccagctgctgtggggagaggccctgctgatcacccccgtgctccaggcaggcaaggcagaggtgacaggctatttccctctgggcacatggtacgacctccagaccgtgccagtggaggccctgggcagcctgcctccaccacctgccgccccccgcgagcctgccatccactccgagggacagtgggtgacactgccagcacctctggacaccatcaacgtgcacctgagggccggctatatcatccccctccagggccctggcctgaccacaaccgagtccagacagcagccaatggccctggccgtggccctgaccaagggaggcgaggccaggggcgagctgttctgggacgatggcgagtctctggaggtgctggagagaggcgcctacacacaggtcatcttcctggccaggaacaatacaatcgtgaatgagctggtgagagtgacctctgagggagcaggactccagctccagaaggtgacagtgctgggagtggcaaccgcaccacagcaggtgctgagcaacggcgtgcccgtgagcaatttcacatactcccctgataccaaggtgctggacatctgcgtgagcctgctgatgggcgagcagtttctggtgtcctggtgttga

SEQ ID NO: 10 - DNA, 1545 bp, synthetic construct
  source 1..1545: mol_type = other DNA
ttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccat

SEQ ID NO: 11 - DNA, 4405 bp, synthetic construct
  source 1..4405: mol_type = other DNA
gaattcgtcgacgccgccaccatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgacccgggatcc

SEQ ID NO: 12 - DNA, 14388 bp, synthetic construct
  source 1..14388: mol_type = other DNA
gatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtctgaccccgtacgcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctgcctgcaggttagaacagtctcttttcgtatgagtgcagtgagtggacgccgccttcgacctgggctgaagaagttcttttctcgaacttcagttcgccggaatacatcattgcccgcacctgtgtcaggctcttagcgccgatatcctggcatgaatgctgaattccggcgatcaggtaaggcacgaatttgtgaatactgcccttatcctggacagctccagacacgccctgtgcgactttgatcttgtctgcctcggaaaaatacctgttctgagaggacagatgcttatccatggcgtccagtgaccccatgcccctatatttcttcagtctgaacccatcactaaagaagtactcgccgggggcttctgtggttgcagccagcaggctgcccatcatcactgtgcttgcccccagagccagggcttttgcgatgtggcccacattctgaattcccccgtcagcgatcactgggactccgaatctccgggcatactcgtacaccttgtagacagcagttgcctgaggtcgtccacaggccagcacttcctgaatgatgcagattgatccactccccattccgaccctcagagcatccactcctgcgtcaatcaggtttttggcctgggctgcggtcacgacattgcctccgatgacctgcagatttgggtacttgtccttaatgtacttgatcatattaatctggaagatgctgtttccctggcttgaatccagcacgaccacgtccacccctgcctgagccagcagatccaggcgatatttatcgtcctcgtgtgtgccaatagcggctccacacagcagctgtttctttgcgtccttactagccagagggtaatctcgatttttcttcaggtcggtgcgggcaatgattgccaccagctcatcgtcttcattcacgataggcagttttcctttcttagaccgctgcagaatctcgttggcttccttcagtgtgatgccggcaggtgcgaccaccagatcttcgcgtttggtcataatctcttccagaaaacagtcatgctcttcctccttcaggaaatcgatgtctcgactagaaatgattcccaccagtcggctgcccattcgtccagtatctgtaatggggatgccgcaaaatccgtgcctagctttggcctcgaacacatcgcggaccctgtccttggggctcaggaccactgggtcggtgataaagccctgttcgtatttcttcacctttctgacctcattggcctgaaattctggagtgcagttatggtgaatgaacccgatcccgcctgtcagtgccatagcaatggccatgccagcctcggtgacagtgtccataggggagctcaccaggggtgtcttcagggtgattttcttggtcagggcagaagtcagatccacctggtctgcggtaaaatcaatatagccgggcaggatcaggaagtcgttgtaagtcagcccgtctccacaattaaacagctgctgggcggtcagtccatcatcagggacataggaagtgcctccagaaatcaggtagtcggccatggtggcgctagccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccaaggaaaaggcgcaaccccaaccccgtggttaattaaggtgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgtcgacgccgccaccatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaatttacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaactcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggacccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaatccacccccagagttgggtgcaccagattgccctgaggatggaggttctgggctgcgaggcacaggacctctactgacccgg